(19) United States
(12) Patent Application Publication
(10) Pub. No.: US 2023/0392121 A1
(43) Pub. Date: Dec. 7, 2023

(54) HYDROGELS FOR CELL CULTURE

(71) Applicant: KATHOLIEKE UNIVERSITEIT LEUVEN, Leuven (BE)

(72) Inventors: Manoj Kumar Gautam, Leuven (BE); Cathérine Verfaillie, Leuven (BE)

(21) Appl. No.: 18/043,405

(22) PCT Filed: Sep. 1, 2021
(86) PCT No.: PCT/EP2021/074179

§ 371 Date: Feb. 28, 2023

(30) Foreign Application Priority Data
Sep. 1, 2020 (EP) 20193913.9
Jul. 2, 2021 (GB) 2109591.4

(51) Int. Cl. C12N 5/071

(52) U.S. Cl. CPC C12N 5/0671; C12N 2506/45; C12N 2513/00; C12N 2533/52; C12N 2533/54

(57) ABSTRACT

The invention relates to compositions comprising a three-dimensional network of a synthetic hydrogel cross-linked with a peptide comprising a metalloproteinase (MMP) cleavable linker characterised in that at least (6) different peptides from an adhesion domain of an Extra Cellular Matrix (ECM) component or from a Cell-Adhesion molecule (CAM) are covalently linked to said hydrogel.

### BACKGROUND OF THE INVENTION

Yearly approximately 2 million people die from liver disease worldwide, half of whom due to liver cirrhosis. Liver cirrhosis is an end-stage liver disease caused by long-standing injury, due to, among others, viral hepatitis, alcohol abuse, non-alcoholic steatohepatitis (NASH), or chronic exposure to chemicals. Liver failure, which is the end-result of cirrhosis, is caused by loss of several critical functions of hepatocytes, including synthesis and secretion of plasma proteins, storage of biomolecules and micronutrients, regulation of glucose homeostasis, metabolism of drugs and blood detoxification. However, hepatocyte loss of function is caused only in part by the direct viral/toxic insult to hepatocytes themselves, but is also amplified by derangements in the cellular interactive network wherein hepatocytes reside. This network includes, among others, hepatic stellate cells (HSCs), liver sinusoidal endothelial cells (LSECs) and liver macrophages (Mkps) (both liver resident Kupffer Cells (KCs), and peripheral blood Mkps recruited to injured liver), all juxtaposed in the liver sinusoid. Therefore, to study the mechanism underlying liver insults, complex test systems incorporating multiple cell types are ideally required. Such models will also be needed to develop efficient therapeutic approaches that can reverse disease processes in and between all cells of this interactive network to prevent or reverse progressive fibrosis causing cirrhosis, progressive hepatocyte loss, and ultimate death of the patient.

To address how toxic insults affect hepatocytes as well as non-parenchymal cells (NPCs), investigators have used in vitro cell culture as well as in vivo rodent models. Complex in vitro liver models can be generated by co-culture of hepatoma cell lines with stellate cell, monocyte, and/or endothelial cell lines. Another approach consists of co-culturing primary human hepatocytes (PHHs) with the NPC fraction from human liver. Such co-cultures can be performed either in stationary cultures or microfluidic organ-on-a-chip systems. Alternatively, precision-cut liver slices have also been used. Although primary liver cell culture systems best mimic the human liver, the main drawbacks of such models are scarcity of human liver cells and the high inter-donor variation of hepatocytes (and, perhaps even more so, NPCs). In addition, murine or rat models are often used to study the effect of different toxic effects on hepatocyte and sinusoidal cell function. However, interspecies differences between human and rodents do not always allow extrapolating findings in animal models to the human patient.

With the advent of pluripotent stem cells (PSC), an alternative approach is to differentiate PSCs to the hepatocyte, stellate cell, endothelial cell and macrophage lineages, and combine these cell populations to create in vitro liver models to mimic the more complex make-up of the in vivo liver. As PSCs are an inexhaustible cell population, this approach should circumvent the inter-donor variation problem observed with primary cells. Moreover, by using a collection of different iPSC lines, it should be possible to create models with known differences in susceptibility to liver damage. Some studies have co-cultured PSC-hepatocyte-like progeny with mesenchymal cells and HUVECs, and more recent studies have created liver models by co-differentiation of PSCs to hepatocyte- and NPC-like cells directly in spheroids. The alternative is to pre-differentiate PSC to hepatocyte-like cells (HLCs) and NPC-like progenitors/cells and ultimately assemble them in co-culture, which is the approach taken in the current study. Hepatoblast-like cells, HSC-like cells, endothelial cells (ECs) and Mφ s from PSCs were generated, which were then combined in a complex co-culture system.

To create multiple-cell co-cultures, different approaches are possible. Cells can be allowed to self-assemble into so-called spheroids, or cells can be embedded in porous scaffolds, such as hydrogels [Hurrell et al. (2020) Cells. 9, 964; Bell et al. (2018) Toxicol Sci. 162, 655-666]. Although the latter is technically more complex, the advantage of hydrogel-based systems is that it is possible to tune the hydrogel to the needs of the microtissue to be created. Natural polymers have often been used for hydrogel creation [Mazzocchi et al. (2018) Biofabrication, 015003. 11(1); Nakai et al. (2019) Biol Open. 8(7), bio042192; Toivonen et al. (2016) Tissue Eng Part A. 22(13-14), 971-984]. However, these polymers are often not well defined and have a limited capacity to represent the ECM diversity of in vivo microenvironments. Therefore, synthetic hydrogels such as polyethylene glycol (PEG) have been used to significantly increase the flexibility of recreating in vivo microenvironments [Stevens et al. (2015) J Biomed Mater Res A. 103(10), 3331-3338]. This includes adjusting the hydrogel to approximate the mechanical properties of a specific organ, as well as mixing with specific extracellular matrix (ECM) components that are typically present in vivo near and around the cells within the organ of interest. The latter can also be replaced by small peptides representing adhesion domains of ECM components or cell-adhesion molecules (CAM), linked to the hydrogel backbone, enabling the creation of a fully tuneable and synthetic hydrogel functionalised to support the needs of specific cells [Lutolf & Hubbell (2003) Biomacromolecules. 4(3), 713-722].

### SUMMARY OF THE INVENTION

The present invention discloses a fully defined hydrogel composition, termed hepatocyte maturation (HepMat) gel, that not only supports maturation of pluripotent stem cell (PSC)-derived hepatocyte-like progeny, but also of the surrounding liver cells, all derived from PSCs, and that is stable for 40 days. The four-cell HepMat-based co-culture system was superior to monocultures of any of the cell populations, in modelling TGP-induced liver fibrosis and fatty acid-induced inflammation and fibrosis. The novel co-culture system has use in for studying mechanisms underlying liver steatosis, inflammation and fibrosis as well as for assessing drugs counteracting these effects.

The invention is summarised in the following statements:

1. A composition comprising a three-dimensional network of a synthetic hydrogel cross-linked with a peptide comprising a metalloproteinase (MMP) cleavable linker characterised in that at least 4 different peptides from an adhesion domain of an Extra Cellular Matrix (ECM) component or from a Cell-Adhesion molecule (CAM) are covalently linked to said hydrogel.

2. The composition according to statement 1, wherein at least 5 or 6 different of said peptides are covalently linked to said hydrogel.

Typically the hydrogel will be functionalised with 6 peptides, it is however envisaged that in certain embodiments 4, 5, or 7 or 8 different peptides are used for functionalising a hydrogel.

3. The composition according to statement 1 or 2, wherein:
the ECM component is selected from the group consisting of Fibronectin, Collagen I, Collagen III, Collagen IV, Laminin, Perlecan, tenascin and Elastin, and
the CAM is E-cadherin or N-cadherin. 4. The composition according to any one of statements 1 to 3, wherein said peptides have a length of between 5, 6, 7, 8, 9 up to 20, 25, 30, 30 or 35 amino acids.

The length of the peptide depends on:
the length of the peptides from the adhesion domain of an Extra Cellular Matrix (ECM) component or from the Cell-Adhesion molecule (CAM)
the length of additional N terminal and C terminal sequences used for crosslinking the peptide and acting as spacer.

5. The composition according to any one of statements 1 to 4, wherein
the peptide of fibronectin comprises an amino acid sequence selected from the group consisting of RGD, RGDS [SEQ ID NO:2], KQAGDV [SEQ ID NO:3], REDV [SEQ ID NO:4], PHSRN [SEQ ID NO:5], and KRLDGS [SEQ ID NO:6],
the peptide of laminin comprises the amino acid sequence YIGSR [SEQ ID NO:8], IKVAV [SEQ ID NO:9] or IKLLI [SEQ ID NO:10],
the peptide of collagen I comprises an amino acid sequence selected from the group consisting of KGHRGF [SEQ ID NO:7], DGEA [SEQ ID NO:11], GTPGPQGIAGQRDVV [SEQ ID NO:12] and GF{Hyp}GER [SEQ ID NO:13],
the peptide of collagen III comprises the amino acid sequence GL{Hyp}GEN [SEQ ID NO:14] or GLKGEN [SEQ ID NO:15],
the peptide of Collagen IV comprises the amino acid sequence TAGSCLRKFSTM [SEQ ID NO:16], FYFDLR [SEQ ID NO:17] or LRRFSTMPFMFCNINNVCNF [SEQ ID NO:24],
the peptide of E cadherin comprises the amino acid sequence LFSHAVSSNG [SEQ ID NO:21] or ADTPPV [SEQ ID NO:23],
the peptide of N cadherin comprises the amino acid sequence HAVDI [SEQ ID NO:22],
the peptide of perlecan comprises the amino acid sequence TWSKVGGHLRPGIVQSG [SEQ ID NO:18],
the peptide of elastin comprises the amino acid sequence VAPG [SEQ ID NO:19], and
the peptide of tenascin comprises the amino acid sequence VAEIDGIEL [SEQ ID NO:20].

6. The composition according to any one of statements 1 to 5, wherein said six different peptides respectively comprise:
a peptide comprising an amino acid sequence of fibronectin,
a peptide comprising an amino acid sequence of collagen I,
a peptide comprising an amino acid sequence of collagen IV, with the proviso of sequence P17,
a peptide comprising an amino acid sequence of Collagen III or laminin, with the proviso of sequence P15 of collagen III, and do not comprise
a peptide comprising an amino acid sequence of perlecan, elastin or tenascin, and
and do not comprise the sequence of SEQ:ID NO: 22 of N cadherin.

The above requirements distinguishes pools 8, 15, 16, 17 and 18 from the other pools in table 2.

The use of peptides pools 8, 15, 16, 17 and 18 give the best performance in BFC metabolisation and CYP3A4 expression of hepatocyte like cells.

7. The composition according to any one of statements 1 to 6, wherein said six different peptides respectively comprise:
a peptide comprising an amino acid sequence of fibronectin,
a peptide comprising an amino acid sequence of laminin,
a peptide comprising an amino acid sequence of collagen I,
a peptide comprising an amino acid sequence of collagen IV,
a peptide comprising an amino acid sequence of E-cadherin, with the proviso of peptide 2,

and do not comprise a peptide comprising an amino acid sequence of perlecan, collagen III, elastin, tenascin or N cadherin.

The above requirements distinguishes pool 15 the other pools in table 2. The use of peptides pool 8, 15, 16, 17 and 18 gives the best performance in BFC metabolisation and CYP3A4 expression of hepatocyte like cells.

8. The composition according to any one of statements 1 to 7, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15),
RGD, [SEQ ID NO:7], [SEQ ID NO:11], [SEQ ID NO:14], [SEQ ID NO:16] and [SEQ ID NO:23] (pool PP16),
[SEQ ID NO:5], [SEQ ID NO:9], [SEQ ID NO:12], [SEQ ID NO:14], [SEQ ID NO:16] and [SEQ ID NO:21] (pool pp17),
[SEQ ID NO:2], [SEQ ID NO:9], [SEQ ID NO:12], [SEQ ID NO:13], [SEQ ID NO:16] and [SEQ ID NO: 24] (pool pp18), and
[SEQ ID NO:6], [SEQ ID NO:10], [SEQ ID NO:11], [SEQ ID NO:13], [SEQ ID NO:16] and [SEQ ID NO:21], (pool pp8).

9. The composition according to any one of statements 1 to 8, wherein said six different peptides comprise respectively the sequence of:
[SEQ ID NO:29], [SEQ ID NO:32], [SEQ ID NO:36], [SEQ ID NO:37], [SEQ ID NO:40] and [SEQ ID NO:47](pool PP15),
RGD, [SEQ ID NO:31], [SEQ ID NO:35], [SEQ ID NO:38], [SEQ ID NO:40] and [SEQ ID NO:47] (pool PP16),
[SEQ ID NO:29], [SEQ ID NO:33], [SEQ ID NO:36], [SEQ ID NO:38], [SEQ ID NO:40] and [SEQ ID NO:45] (pool pp17),
[SEQ ID NO:26], [SEQ ID NO:33], [SEQ ID NO:36], [SEQ ID NO:37], [SEQ ID NO:40] and [SEQ ID NO:48] (pool pp18), and
[SEQ ID NO:30], [SEQ ID NO:34], [SEQ ID NO:37], [SEQ ID NO:37], [SEQ ID NO:40] and [SEQ ID NO:45] (pool pp8).

10. The composition according to any one of statements 1 to 9, wherein said six different peptides comprise respectively the sequence of [SEQ ID NO:5], [SEQ ID NO:8], [SEQ ID NO:12], [SEQ ID NO:13], [SEQ ID NO:16] and [SEQ ID NO:23](pool PP15).

11. The composition according to any one of statements 1 to 10, wherein said six different peptides comprise respectively the sequence of [SEQ ID NO:29], [SEQ ID NO:32], [SEQ ID NO:36], [SEQ ID NO:37], [SEQ ID NO:40] and [SEQ ID NO:47](pool PP15).

12. The composition according to any one of statements 1 to 11, wherein the synthetic hydrogel is Polyethylene glycol.

Another suitable synthetic hydrogel is polyacryl amide.

13. The composition according to any one of statements 1 to 12, wherein PEG is at a concentration 5-15 or of 8-12% hydrogel/composition.

14. The composition according to any one of statements 1 to 13, wherein the PEG is a 4-armed PEG with Mr of 10.000 kD.

In alternative embodiments, PEG is a 3-arm PEGs, a 6-arm PEGs, a 8-arm PEGs or a Y-Shape PEGs.

PEG Mr can range from 5.000, 7.500, 10.000 to 15.000, 20.000, 25.000.

15. The composition according to any one of statements 1 to 14, wherein the composition has a stiffness from 2, 3, 5, 10 to 20, 25, 30, 40 or 50 KPa, e.g. between 3 and 30 KPa.

16. The composition according to any one of statements 1 to 15, wherein the metalloproteinase (MMP) cleavable linker comprises the sequence GPQGIAGQ [SEQ ID NO:52].

17. A composition in accordance with any one of statements 1 to 16, and further comprising PSC differentiated into hepatocyte-like cells and optionally one or more of hepatic stellate cells, liver sinusoidal endothelial cells and liver macrophages.

18. The composition according to statement 17, comprising PSC differentiated into hepatocyte-like cells, hepatic stellate cells (HCS), liver sinusoidal endothelial cells (LSECS) and liver macrophages (Mphis).

19. The composition according to statement 17 or 18 wherein
the hepatocyte like cells metabolise 7-Benzyloxy-4-triFluoromethyl-Coumarin (BFC), or express one or more of HNF4a, HNF6, PROX1, CYP3A4, CYP2D6, PEPCK, G6PC and PGC1a.
the hepatic stellate cells express one or more of PDGFR-alpha, DESMIN, LRAT, RGS5, COL3A1, COL1A1, LOXL2 and NCAM,
the liver sinusoidal endothelial cells express one or more of FCGR2B, STAB1, FCN3, OIT3 and CLEC4G, and
the liver macrophages express one or more of MARCO, CD5L, SIGLEC1, CD163, CD45, CD68, CD14.

20. The composition according to statements 17 or 18, wherein said cells are aggregated into spheroids.

21. The composition according to statement 20, wherein the spheroids comprise duct like structures and/or tubular structures of ECS surrounded by HSC cells.

22. The composition according to any one of statements 1 to 20, which comprises a cell cultivation medium with a glycine concentration of at least 20 mg/ml.

23. Use of a composition in accordance to any one of statements 17 to 22, for toxicity testing of compounds or conditions applied on said cells.

24. Use of a composition in accordance to any one of statements 17 to 22, for inducing an fibrogenic cell type by TGFbeta or inducing an inflammatory cell type by oleic acid.

25. Use of a composition in according with any one of statements 1 to 24, for cultivating liver organoids.

Another aspect of the present invention is a method wherein a platform is presented wherein other functionalised hydrogels are tested for their capacity of maintaining the hepatocyte phenotype of the cells.

26. A method of identifying a composition suitable for cell growth of PSC cells differentiated into hepatocyte like cells, comprising the steps of:
Providing a cell culture medium comprising an artificial matrix, wherein said matrix comprises at least 4 different peptides of an adhesion domain from an Extra Cellular Matrix (ECM) component or from an Cell-Adhesion molecule (CAM), covalently linked to a hydrogel,
Growing PSC cells in said medium under conditions allowing the differentiation of said PSC cells into hepatocyte cells,
testing a one or more parameters indicative of a hepatocyte like phenoype,
based on said one or more parameters, identifying a matrix as suitable for cultivation of hepatocyte like cells differentiated from PSC.

27. The method according to statement 26, wherein said matrix comprises at least 4 different peptides of an adhesion domain from an Extra Cellular Matrix (ECM) component or from an Cell-Adhesion molecule (CAM)

28. The method according to statement 26 or 27, wherein at least 5 or 6 different of said peptides are covalently linked to said hydrogel.

29. The method according to any one of statements 26 to 28, wherein
the ECM component is selected from the group consisting of Fibronectin, Collagen I, Collagen III, Collagen IV, Laminin, Perlecan, tenascin and Elastin, and
wherein the CAM is E-cadherin or N-cadherin.

30. The method according to any one of statements 26 to 29, wherein said peptides comprise a sequence selected from the group consisting of RGD and the peptides with SEQ ID NO:2 to SEQ ID NO:24.

31. The method according to any one of statements 26 to 30, wherein the synthetic hydrogel is cross-linked with a peptide comprising a metalloproteinase (MMP) cleavable linker.

32. The method according to statement 31, wherein the metalloproteinase (MMP) cleavable linker comprises the sequence GPQGIAGQ [SEQ ID NO:52].

33. The method according to any one of statements 26 to 32, wherein the synthetic hydrogel is Polyethylene glycol (PEG).

34. The method according to any one of statements 26 to 32, wherein the matrix comprises 8-12% (wt/v) PEG.

35. The method according to statement 33 or 34, wherein the PEG is a 4-armed PEG with Mr of 10.000 kD.

36. The method according to any one of statements 26 to 35, wherein the matrix has a stiffness of between 3 and 30 Kpa.

37. The method according to any one of statements 26 to 36, and further comprising the steps of:
growing one or more of hepatic stellate cells, liver sinusoidal endothelial cells and liver macrophages and testing one or more parameters indicative for the phenotype of said cell, and,
based on said one or more parameters, identifying a matrix as suitable for co-cultivation of hepatocyte like cells with one or more of hepatic stellate cells, liver sinusoidal endothelial cells and liver macrophages.

38. The method according to statement 37 wherein:
a parameter indicative of a hepatocyte like phenoype is the metabolisation of 7-Benzyloxy-4-triFluoromethyl-Coumarin (BFC), or the expression of one or more of HNF4a, HNF6, PROX1, CYP3A4, CYP2D6, PEPCK, G6PC and PGC1a,
a parameter indicative of hepatic stellate cells is the expression of one or more of PDGFR-alpha, DESMIN, LRAT, RGS5, COL3A1, COL1A1, LOXL2 and NCAM,
a parameter indicative of liver sinusoidal endothelial cells is expression of one or more of FCGR2B, STAB1, FCN3, OIT3 and CLEC4G, and,
a parameter indicative of liver macrophages is expression of one or more of MARCO, CD5L, SIGLEC1, CD163, CD45, CD68, CD14.

39. The method according to statement 38, which is performed in a cell cultivation medium comprising at least 20 mg glycine/ml medium.

40. A composition comprising a three-dimensional network of a synthetic hydrogel covalently cross-linked with at least 6 different peptides, each peptide comprising a metalloproteinase (MMP) cleavable linker and fragment from an adhesion domain of an Extra Cellular Matrix (ECM) component or from a Cell-Adhesion molecule (CAM), wherein said at least six different peptides comprise:
a peptide from fibronectin,
a peptide from collagen I,
a peptide from collagen IV, other than the collagen IV fragment FYFDLR [SEQ ID NO:17],
a peptide from Collagen III, other than collagen III fragment GLKGEN [SEQ ID NO:15],
a peptide from laminin, and wherein said at least six peptides do not comprise a peptide from perlecan, from elastin or from tenascin,
and do not comprise the peptide HAVDI [SEQ ID NO:22] of N cadherin.

41. The composition according to statement 40, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
RGD, [SEQ ID NO:7], [SEQ ID NO:11], [SEQ ID NO:14], [SEQ ID NO:16] and [SEQ ID NO:23] (pool PP16), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18), or
[SEQ ID NO:6], [SEQ ID NO:10], [SEQ ID NO:11], [SEQ ID NO:13], [SEQ ID NO:16] and [SEQ ID NO:21] (pool pp8).

Explicitly disclosed herein are hydrogels with any one of the above mentioned 6 pools individually and any combination of 2, 3, 4, or 5 of the above mentioned pools.

Preferred embodiments are:

41. The composition according to statement 40, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18).

41. The composition according to statement 40, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
RGD, [SEQ ID NO:7], [SEQ ID NO:11], [SEQ ID NO:14], [SEQ ID NO:16] and [SEQ ID NO:23] (pool PP16), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18).

41. The composition according to statement 40, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
RGD, [SEQ ID NO:7], [SEQ ID NO:11], [SEQ ID NO:14], [SEQ ID NO:16] and [SEQ ID NO:23] (pool PP16), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18), or
[SEQ ID NO:6], [SEQ ID NO:10], [SEQ ID NO:11], [SEQ ID NO:13], [SEQ ID NO:16] and [SEQ ID NO:21] (pool pp8).

41. The composition according to statement 40, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
RGD, [SEQ ID NO:7], [SEQ ID NO:11], [SEQ ID NO:14], [SEQ ID NO:16] and [SEQ ID NO:23] (pool PP16), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18), or
[SEQ ID NO:6], [SEQ ID NO:10], [SEQ ID NO:11], [SEQ ID NO:13], [SEQ ID NO:16] and [SEQ ID NO:21] (pool pp8), or
[SEQ ID NO:3], [SEQ ID NO:7], [SEQ ID NO:9], [SEQ ID NO:12], [SEQ ID NO:13], and [SEQ ID NO:18] (pool pp9).

42. The composition according to statement 40 or 41, wherein said six different peptides comprise respectively the sequence of [SEQ ID NO:5], [SEQ ID NO:8], [SEQ ID NO:12], [SEQ ID NO:13], [SEQ ID NO:16] and [SEQ ID NO:23] (pool PP15).

43. The composition according to any one of statements 40 to 42, wherein the synthetic hydrogel is a 4-armed polyethylene glycol PEG with Mr of 10.000 kD.

44. The composition according to any one of statements 40 to 43, wherein the metalloproteinase (MMP) cleavable linker comprises the sequence GPQGIAGQ [SEQ ID NO:52].

45. A composition in accordance with any one of statements 40 to 44, and further comprising PSC differentiated into hepatocyte-like cells and optionally one or more of hepatic stellate cells, liver sinusoidal endothelial cells and liver macrophages.

46. The composition according to statement 45, wherein:
the hepatocyte like cells metabolise 7-Benzyloxy-4-triFluoromethyl-Coumarin 30 (BFC), or express one or more of HNF4a, HNF6, PROX1, CYP3A4, CYP2D6, PEPCK, G6PC and PGC1a.
the hepatic stellate cells express one or more of PDGFR-alpha, DESMIN, LRAT, RGS5, COL3A1, COL1A1, LOXL2 and NCAM,
the liver sinusoidal endothelial cells express one or more of CD31, FCGR2B, STAB1, FCN3, OIT3 and CLEC4G, and
the liver macrophages express one or more of MARCO, CD5L, SIGLEC1, CD163, CD45, CD68, CD14.

47. The composition according to statement 45 or 46, wherein said cells are aggregated into spheroids.

48. Use of a composition in accordance to any one of statements 45 to 47, for toxicity testing of compounds or conditions applied on said cells.

49. Use of a composition in accordance to any one of statements 45 to 48, for the cultivation of PSC differentiated into hepatocyte-like cells and cultivation of optionally one or more of hepatic stellate cells, liver sinusoidal endothelial cells and liver macrophages.

50. A method of identifying a matrix suitable for cell growth of PSC cells differentiated into hepatocyte like cells, comprising the steps of:
Providing a cell culture medium comprising an artificial matrix, wherein said matrix comprises at least 4 different peptides of an adhesion domain from an Extra Cellular Matrix (ECM) component or from an Cell-Adhesion molecule (CAM), covalently linked to said hydrogel, wherein the peptides further comprise a MMP cleavable sequence,
Growing PSC cells in said medium under conditions allowing the differentiation of said PSC cells into hepatocyte cells,
testing a one or more parameters indicative of a hepatocyte like phenotype,
based on said one or more parameters, identifying a matrix as suitable for cultivation of hepatocyte like cells differentiated from PSC.

51. The method according to statement 50, wherein
the ECM component is selected from the group consisting of Fibronectin, Collagen I, Collagen III, Collagen IV, Laminin, Perlecan, tenascin and Elastin, and
wherein the CAM is E-cadherin or N-cadherin.

52. The method according to statement 50 or 51, and further comprising the steps of:
growing one or more of hepatic stellate cells, liver sinusoidal endothelial cells and liver macrophages and testing one or more parameters indicative for the phenotype of said cell, and,
based on said one or more parameters, identifying a matrix as suitable for co-cultivation of hepatocyte like cells with one or more of hepatic stellate cells, liver sinusoidal endothelial cells and liver macrophages.

53. The method according to statement 52 wherein:
a parameter indicative of a hepatocyte like phenotype is the metabolisation of 7-Benzyloxy-4-triFluoromethyl-Coumarin (BFC), or the expression of one or more of HNF4a, HNF6, PROX1, CYP3A4, CYP2D6, PEPCK, G6PC and PGC1a,
a parameter indicative of hepatic stellate cells is the expression of one or more of PDGFR-alpha, DESMIN, LRAT, RGS5, COL3A1, COL1A1, LOXL2 and NCAM,
a parameter indicative of liver sinusoidal endothelial cells is expression of one or more of CD31, FCGR2B, STAB1, FCN3, OIT3 and CLEC4G, and,
a parameter indicative of liver macrophages is expression of one or more of MARCO, CD5L, SIGLEC1, CD163, CD45, CD68, CD14.

### DETAILED DESCRIPTION

### Figure Legends

FIG. 1. (A-C) Identification of optimal mechanical properties and peptide functionalisation of polyethylene glycol (PEG) hydrogels to support maturation of PSC-hepatocyte like cells (HLCs).

(A) Analysis of DOE experiment using the Fit Two Level Model- and Half Normal Plot Model-based analysis from the JMP-pro package based on transcript levels for 8 hepatocyte specific genes and BFC metabolisation to define the degradability and stiffness of the hydrogel that most optimally supports HLC maturation.

(B-C) Analysis of DOE to identify the hydrogel characteristics (peptide pool, MMP linker and PEG concentration) that supports the highest maturation of PSC-HLCs (based on transcript levels for 8 hepatocyte specific genes and BFC metabolisation): Fit Two Level Model B and Reference Scaled Average analysis C MP1, MP2, MP3=different PEG concentrations; DG1, DG2, DG3=different MMP cleavable linkers; DOE=design of experiment; BFC=7-benzyloxy-4-trifluoromethylcoumarin; PHH=primary human hepatocytesration of PSC-HLCs. (D-E) Confirmatory screens confirm that the MP2-DG2-PP15 hydrogel optimally supports HLC differentiation, both in basic liver differentiation medium (LDM) and amino-acid-glycine supplemented (AAGly) medium.

(D) Bulk mechanical properties measured by nanoindentation of hydrogels containing 3 different concentrations of PEG polymers, cross-linked with (P26), and functionalised with 6 different functionalising peptides (N=2 biological replicates).

(E) Fluorescent intensity measurements obtained from fluorescent 5-FAM peptide conjugated PEG hydrogels at different concentrations. Each dot is representing different planes in z-axis per group (N=2 biological replicates). MP2=PEG concentration; DG2=MMP cleavable linkers; PHH=primary human hepatocytes; LDM=liver differentiation medium; AAGly medium is LDM+extra amino acids.

FIG. 2. HepMat hydrogel supports further maturation of PSC-HLC progeny (derived from TF (HC3x) overexpressing PSC and amino acid supplemented medium (AAGly), as described in Boon et al. (2020) Nat Commun. 11(1), 1393.

(A) Gene expression of HNF4a, HNF6, CYP3A4, CYP2D6, G6PC, PEPCK and PGC1a in HC3x-progeny maintained in 2D culture for 40 days vs. progeny of d8 2D-culture derived hepatoblasts embedded in HepMat hydrogels for an additional 32 days (N=3 biological replicates).

(B) Functional analysis of day 40 HC3x-progeny in 2D or 3D-HepMat cultures: Upper panel: CYP3A4 function defined by BFC metabolisation (2D: N=5; 3D: N=13; 3D-RIF: same as 3D but cells were exposed to 25 μM of Rifampicin (RIF) for 48 h, N=4; PHH, N=3; N=biological replicates); Lower panel: Albumin secretion (N=3 biological replicates) Data are shown as mean±SEM and analysed by two-tailed student t-test (RT-qPCR) or one way Anova (Tukey's multiple comparison for BFC). *p<0.05; **p<0.01; Scale bars=100 mm; PHH=primary human hepatocytes.

(C) Histology of day 40 HC3x-progeny in 3D HepMat cultures: H & E staining of cells; (Representative examples of 3 biological replicates).

(D) HepMat hydrogel supports maturation of PSC-HLC progeny (derived from TF (HC3x) overexpressing PSC, albeit less robustly in LDM medium compared to AAGly medium. HC3x-PSCs were cultured either for 40 days in 2D culture or initially for 8 days in 2D culture and then in HepMat hydrogels for 32 days using liver differentiation medium without extra amino acids [Boon et al. cited above]. Gene expression of HNF4a, HNF6, CYP3A4, CYP2D6, G6PC, PEPCK and PGC1a in day 40 HC3x-progeny maintained in 2D culture vs. progeny from HepMat hydrogel cultures s (N=3 biological replicates).

Data are shown as mean±SEM and analysed by two-tailed student t-test; *p<0.05; **p<0.01.

FIG. 3. The HepMat hydrogel also maintains PSC non-parenchymal cells for at least 32 days.

(A) PSC-endothelial cells (ECs): RT-qPCR for the general EC marker gene, CD31, and LSEC marker genes (FCGR2B, STAB1, FCN3, LYVE1 and MRC1) in day 12 2D iETV2-progeny or recovered after an additional 32 days of culture in HepMat hydrogel (N=3 biological replicates).

(B) RT-qPCR for HSC marker genes (ACTA2, COL1A1, COL5A2 and LRAT) in day 12 2D HSC-progeny or recovered after an additional 32 days of culture in HepMat hydrogel (N=3 biological replicates). Treatment of PSC-HSC cultured in HepMat hydrogels for 32 days with 25 ng/mL TGFβ for an additional 24 h: Relative expression for HSC marker genes (COL1A1, COL3A1, LRAT) and pro-collagen secretion (by ELISA) in TGFβ-treated and non-treated cells (N=3 biological replicates).

(C) PSC-macrophages (MO) RT-qPCR for MO/KC marker genes (CD68, MARCO, CD5L, SIGLEC1) in day 12 2D MO-progeny or recovered after an additional 32 days of culture in HepMat hydrogel (N=3 biological replicates). Treatment of MO embedded in HepMat hydrogels for 32 days with 100 ng/mL LPS for an additional 24 h: Relative inflammatory marker gene expression (TNF-α, IL-1a, IL6) and TNF-α, IL-1a, IL6 secretion (by ELISA) in LPS-treated and non-treated cells (N=3 biological replicates).

Data are shown as mean±SEM and analysed by two-tailed student t-test. *p<0.05; **p<0.01; ***p<0.001; ****p<0.0001. Scale bars=100 μm.

(D) PSC-endothelial cells (ECs): iETV2-PSCs were differentiated in 2D to ECs, RT-qPCR for EC/LSEC marker genes (CD31, FGFR2B, STAB1, CLEC4G) on d8 and d12 of 2D differentiation (N=3 biological replicates). FACS analysis for CD31 and KDR on day 8 of 2D culture (representative for N=2 biological replicates). RT-qPCR for the LSEC marker genes (OIT3, CLEC4G and CLEC4M) in ETV2-progeny harvested after 12 days of differentiation in 2D cultures or recovered after an additional 32 days of culture in HepMat hydrogel (N=3 biological replicates).

(E) PSC-hepatic stellate cells (HSCs): RT-qPCR for HSC marker genes (RGS5, IGFBP5, PDGFRα, COL3A1 and LOXL2) in PSC-HSCs after 12 days of differentiation in 2D cultures or 12 days in 2D culture and additional 32 days of culture in HepMat hydrogel (N=3 biological replicates).

(F) PSC-Macrophages (MO): RT-qPCR for Mφ marker genes (CD168, CD14, CD45) in PSC-Mφ after 16 days of differentiation in 2D culture or 16 days in 2D culture and 32 days of culture in HepMat hydrogel (N=3 biological replicates).

Data are shown as mean±SEM and analysed by two-tailed student t-test. **p<0.01; ***p<0.001; ****p<0.0001.

FIG. 4. HepMat hydrogel maintains and supports maturation/quiescence of PSC-hepatocyte-like cells, -endothelial cells, -hepatic stellate cells and -macrophages for at least 32 days.

(A) RT-qPCR for marker genes for the different cell types: HNF6, HNF4a, CYP3A4, CYP2D6, G6PC, PEPCK and PGC1a as HLC markers; CD31, FCGR2B, STAB1, LYVE1, MRC1 and CLEG4M as general EC or LSEC markers, PDGFRα, COL5A2, COL1A1, COL3A1, ACTA2 and LRAT as PSC-HSC markers and MARCO, CD5L, SIGLEC1 and CD68 PSC-MΦ/KC markers in cells harvested after 32 days from respective 3D mono-cultures (3D) or 3D co-cultures (co-culture)(N=3 biological replicates).

Data are shown as mean±SEM and analysed by two-tailed student t-test.*p<0.05; **p<0.01; ***p<0.001.

(B) CYP3A4 function defined by BFC metabolisation (3D=monoculture of HC3x-HLCs in HepMat hydrogel and liver differentiation medium, N=4; co-culture and co-culture-RIF=HC3x-PSC, ETV2-PSCs, PSC-HSCs and PSC-MΦ progeny co-culture without and with 25 μM of Rifampicin (RIF) for 48 h, N=15 and N=6, resp.; PHH; N=) (C) The HepMat hydrogel maintains and supports maturation/quiescence of PSC-hepatocyte-like cells, -endothelial cells, -hepatic stellate cells and -macrophages for at least 32 days. Additional RT-qPCR data for marker genes for the different cell types:CLEC4G, FCN3 and OIT3 as general EC or LSEC markers, RGS5, IGFBP5 and LOXL2 as PSC-HSC markers and MARCO, CD5L, SIGLEC1 and CD68 PSC-MΦ/KC markers in cells harvested after 32 days from respective 3D mono-cultures (3D) or 3D co-cultures (co-culture)(N=3 biological replicates).

(D) Multiple Iterative Labelling by Antibody Neodeposition (MILAN) analysis of 32 day HepMat hydrogels seeded with a combination of HC3x-hepatoblasts, ETV2-ECs, PSC-HSCs and PSC-MΦs. Hydrogels were fixed without collagenase degradation, paraffin embedded and sectioned The table shows an overview of all cell types present obtained with the MILAN analysis after processing of the immunofluorescence images.

(E) expression level per cell group for each of the included markers in multiplex analysis

FIG. 5. HepMat-based co-cultures of HC3x-hepatocyte-like cells, iETV2-endothelial cells, PSC-hepatic stellate cells and PSC-macrophages can be used to model liver fibrosis.

Relative expression of fibrogenic markers (LOXL2, COL1A1, COL3A1 and COL5A2) and pro-inflammatory cytokines (TNF-α, IL-1a and IL6) in d32 HepMat 4-cell co-cultures or PSC-HC3x-HLC, PSC-HSC or PSC-MΦ monocultures exposed to TGFβ for 3 and 7 days (N=3 biological replicates)(horizontal dotted line represents the control sample). (B) Relative measurement of secreted protein pro-collagen and pro-inflammatory cytokines (TNF-α, IL-1α and IL6) in d32 HepMat 4-cell co-cultures or PSC-HC3x-HLCs, PSC-HSCs or PSC-MΦ mono-cultures exposed to TGFβ for 3 and 7 days (N=3 biological replicates) (horizontal dotted line represents the control sample).

Data are the mean±SEM (N=3) and analysed by one-way Anova (Tukey's multiple comparison).*p<0.05; **p<0.01; ***p<0.001, ****p<0.0001; N.D.: Not detected.

FIG. 6. HepMat-based co-cultures of PSC-hepatocyte-like cells, -endothelial cells, -hepatic stellate cells and -macrophages can be used to model liver fibrosis and steatosis.

(A) Relative expression of fibrogenic markers (LOXL2, COL1A1, COL3A1 and COL5A2) and pro-inflammatory cytokines (TNF-α, IL-1α, and IL6) in d32 HepMat 4-cell co-cultures or PSC-HC3x-HLCs, PSC-HSCs or PSC-MΦ monocultures exposed to OA for 3 or 7 days (N=3 biological replicates)(horizontal dotted line represents the control sample).

(B) Relative measurement of secreted protein (by ELISA) for pro-collagen and pro-inflammatory cytokines (TNF-α, IL-1α and IL6) in d32 HepMat 4-cell co-cultures or PSC-HC3x-HLCs, PSC-HSCs or PSC-MΦ mono-cultures exposed to OA for 3 or 7 days (N=3 biological replicates)(horizontal dotted line represents the control sample).

(C) Relative expression of fibrogenic and proinflammatory marker genes, and secreted pro-collagen, TNF-α, IL-1α and IL6 in d32 HepMat 4-cell co-cultures exposed to BSA, OA alone, OA&OCA or OA&ELN for 3 days (N=3 biological replicates)(horizontal dotted line represents the control sample)

Data are the mean±SEM (N=3) and analysed by one-way Anova (Tukey's multiple comparison).*p<0.05; **p<0.01; and ***p<0.001, ****p<0.0001. OA: Oleic acid; OCA: Obeticholic acid; ELN: Elafibranor; N.D.: Not detected.

(D) data of representative confocal fluorescence images of d32 HepMat co-cultures exposed for 3 days to BSA as control, oleic acid (OA; 800 μM), a combination of oleic acid+obetecholic acid (OA; 800 μM+OCA; 1 μM) or oleic acid+Elafibranor (OA; 800 μM+ELN; 30 μM); Relative Bodipy intensity compared with BSA control (N=3 biological replicates).

Using a design-of-experiment (DOE) approach, 216 different compositions of hydrogels functionalised with different combinations of ECM- and CAM-peptides, cross-linked with different metalloproteinase (MMP)-cleavable linkers and of variable stiffness were tested. A specific hydrogel composition was identified that supported maturation of PSC-HLCs, which was termed “hepatocyte maturation” or “HepMat” hydrogel. As explained in more detail HepMat is an embodiment of the present invention wherein a PEG hydrogel is cross-linked with a MMP biodegradable peptide. Peptides with SEQ ID: 25-48 are covalently linked to the PEG hydrogel.

This HepMat hydrogel was shown to also support the survival of three PSC-derived NPC populations, namely HSC-like cells, ECs and Mps. Upon co-culture of the PSC-NPCs and PSC-HLCs in HepMat hydrogels, all cells survived for up to 40 days, and self-assembled into spheroids consisting of cells with a hepatocyte phenotype (more mature and immature hepatocytes) interspersed with cells with an EC, HSC or Mφ phenotype, small bile duct-like structures, and tubular structures consisting at least in part ECs and mesenchymal cells. Compared with prior art 2D cultures, the 3D-HepMat co-cultures supported further maturation of HLCs, including a fraction of cells that no longer stained positive for AFP, and may induce an apparent less fibrogenic phenotype of HSCs than in 2D cultures. Finally, the four-cell co-culture system is superior to monocultures for testing the ability of TGFβ or oleic acid (OA) to induce a fibrogenic and inflammatory cell phenotype, which could be blocked at least in part by treatment with obeticholic acid and to a lesser degree elafibranor.

The present invention describes the creation of a fully tuneable and synthetic PEG-based hydrogel, functionalised with a combination of ECM and CAM binding sequences (termed hepatocyte maturation hydrogel, or HepMat hydrogel) that supports the maintenance and maturation of PSC-HLCs progeny. In line with the hypothesis that such a hydrogel composition also allows maintenance of NPCs, co-localised in vivo with hepatocytes in liver sinusoids, it is demonstrated herein that HSCs and Mps generated from PSCs survive in the HepMat hydrogel for at least 32 days, while maintaining their fibrogenic response ability to TGFβ and inflammatory response to LPS, respectively. PSC-ECs (generated by overexpression of the master regulator ETV2 [Elcheva et al. (2014) Nat Commun. 5, 4372], could also be maintained for at least 32 days, even if tube formation ceased from day 15 onwards. Co-culture of PSC-HLCs and PSC-NPCs in a 4-cell type HepMat-co-culture system further enhanced HLC maturation, supported persistent vessel-like structure establishment and possible fating of ECs to cells with a more LSEC-like phenotype, induced a less activated state of HSCs, and apparently induced some KC like features in Mφs. Most importantly, the 4-cell co-culture had a far greater fibrogenic and inflammatory response to TGFβ and, even more so, OA, than any of the prior mono-cultures, attesting to the requirement of an intricate interaction between NPCs and HLCs in the development of liver fibrosis and inflammation, and the capability of the HepMat-PSC-HLC-NPC co-culture to model liver fibrosis/inflammation. The pro-inflammatory and -fibrogenic effects of OA could be blocked by treatment of the 4 cell-type HepMat co-culture with obeticholic acid. The culture system is thus of use in developing anti-NASH/fibrogenic drugs.

It has been long-established that when cultured on stiff culture plates, PHHs very quickly lose functionality. This is also the case for HSCs that become quickly activated, and LSECs that very quickly lose their typical sinusoidal endothelial characteristics. Especially for hepatocytes, numerous studies have demonstrated that this can at least in part be prevented when cells are cultured for instance in sandwich cultures in spheroids or embedded in scaffolds [Hurrell et al. cited above; Mazzocchi et al. cited above; Nakai et al. cited above; Toivonen et al. cited above; Godoy et al. (2016) Arch Toxicol. 90, 12513-12529; Jaramillo (2018) J Tissue Eng Regen Med. 12(4), e1962-e1973; Prestwich (2011) J Control Release. 155(2), 193-199; Ma et al. (2016) Proc Nat/Acad Sci USA. 113(8), 2206-2211]. Scaffolds used range from decellularised liver scaffolds to natural polymer-based scaffolds, synthetic scaffolds [Stevens et al. cited above; Christoffersson (2018) Biofabrication. 11(1), 015013; Mobarra et al. (2019) J Cell Physiol. 234(7), 11247-55; Wang et al. (2003) Biomaterials. 24(19), 3213-3220], or hybrid natural/synthetic hydrogels [Prestwich (2011) cited above; Ma et al. (2016) Proc Natl Acad Sci USA. 113, 2206-2211].

In initial studies, the maturation of HLCs was tested in different natural polymers, by embedding day 8 hepatoblasts in collagen, matrigel, and or gelatin gels for up to 28 days. Although culture in a mixture of gelatin and matrigel increased some hepatic marker transcripts, the hydrogels were rapidly degraded, and could not be used for long-term maintenance of PSC-HLCs. As classical natural polymers also suffer from batch-to batch variability, even if this might be overcome by using for instance synthetically created polymers [Kanninen et al. (2016) Biomaterials 103, 86-100], PEG-based gels were cross-linked in a non-cytotoxic manner with linkers that can be cleaved by MMPs thought to be present in liver. Linkers were chosen that would be relatively slowly degraded to ensure stability of the hydrogel for at least 30 days [Patterson et al. (2010) Materials Today. 13, 14-22]. As PEG is inert and does not allow cell adhesion, the PEG gel can be mixed with natural polymers. However, again the batch-to-batch variability of the natural polymers may prevent creation of highly consistent culture conditions. Peptide ligands were linked representing cell adhesion domains of ECM molecules and or CAMs, present human liver to the PEG hydrogel [Naba et al. (2016) Matrix Biol. 49, 10-24].

A DOE approach was approached to enable screening the innumerable combinations of functionalised hydrogels, based on PEG concentration and degradability by MMP-cleavage and the 24 different peptides selected. The DOE narrowed down the number of combinations to 216 different environments, whereas a non-DOE approach would have required screening of 23.328 conditions. A four-arm PEG at a concentration of 10% w/v, cross linked with the MMP9 cleavable linker peptide, GCRDGPOGIAGQDRCG [SEQ ID NO:50], and functionalised by peptides comprising sequences of fibronectin [P5][SEQ ID NO:5], laminin [P8] [SEQ ID NO:8], collagen I [P12 and P13] [SEQ ID NO:12] and [SEQ ID NO:13], collagen IV [P16] [SEQ ID NO:16] and E-cadherin [P23] [SEQ ID NO:23] (Table 1) generated PSC-HLC that clustered transcriptionally and functionally significantly closer to PHHs than 2D cultured PSC-HLCs. Some hydrogel compositions however had detrimental effects on HLC maturation.

Significantly improved CYP450 activity, drug biotransformation and cellular metabolism of PSC-HLCs was obtained in the prior art when three hepatic transcription factors HNF1A, FOXA3 and PROX1 are overexpressed and these cells (called HC3x cells) were matured in amino acid rich (AAGly) medium in 2D cultures [Boon et al. cited above]. AAGly is a cell culture medium comprising 3.7 mg amino acid/ml and a further 20 mg/ml glycine as detailed in Boon et al. When the same HC3X progeny is maintained in 3D HepMat hydrogel of the present invention and supplemented with AAGly medium, a significant further transcriptional and functional maturation can be obtained compared to the prior art 2D cultures. This included a significant induction of genes involved in gluconeogenesis and mitochondrial biogenesis and significantly enhanced and rifampicin-inducible CYP3A4 activity. However, when HC3x-HLCs were maintained in HepMat hydrogels in baseline LDM medium not supplemented with extra AAs, HLC functionality remained significantly inferior to the HepMat-AAGly culture condition, demonstrating the contribution of both the functionalised hydrogel and AA support for HLC maturation.

Since hepatocytes and NPCs are juxtaposed in vivo in the liver sinusoid, hydrogel compositions that support long-term stable maturation of HLCs would also support different cells co-inhabiting the hepatocyte niche in liver sinusoids. This is confirmed, as the HepMat formulation could maintain PSC-HSC (as described in Coll et al. (2018) Cell Stem Cell. 23, 101-13 e7] for at least 32 days. Culture of HSCs in the HepMat hydrogel induced a transcriptional profile more reminiscent of non-fibrotic HSCs, even if not all gene markers recently described in Ramachandran et al. (2019) Nature 575, 512-518, were induced. This is consistent with the notion that culture of HSCs in 3D may counteract activation observed when cells are cultured on stiff surfaces [Mannaerts et al. (2015) J Hepatol. 63(3), 679-688; Van Grunsven (2017) Adv Drug Deliv Rev. 121, 133-146]. Noteworthy, a fibrogenic response of HSCs, embedded in the HepMat hydrogel was still feasible, as shown by incubation with TGFβ. This gene induction far exceeded what was previously reported for TGFβ treated PSC-HSCs (or primary human HSCs) cultured in 2D culture, likely because of the significantly less fibrotic phenotype of HepMat cultured HSCs compared with 2D cultured cells. This indicates that PSC-HSCs cultured in the 3D-HepMat hydrogel are a suitable model, in itself, to study HSC activation and examine drugs that might counteract fibrogenic responses. In addition, the HepMat hydrogel supported survival of PSC-ECs for 32 days, even if spontaneous tube formation ceased after 15 days. This is likely due to the absence of cells, such as mural cells, known to stabilise EC tube formation in vivo and in vitro in these mono-cultures. Finally, HepMat hydrogels also supported the survival of PSC-Mφs for at least 32 days beyond the 16+ days of culture in 2D suspension differentiation cultures, and, even following 32 days in hydrogel culture, PSC-Mφs could be activated by LPS to produce inflammatory cytokines. As the ECM/CAM interaction requirements of some/all of the NPCs may differ in some aspects from that of hepatocytes, it is an aspect of the present invention to develop alternatives of the HepMat hydrogel that are functionalised with additional ECM/CAM peptides, or wherein other combinations of peptides and/or hydrogel and/or cleavable linkers are used for an even better support of the different NPCs.

All four PSC-derived cell populations were co-incorporated in the HepMat gel to create an all-PSC derived 3D liver model, and the model was tested for its suitability to study liver inflammation and fibrosis. The AAGly supplementation used for optimal differentiation of 2D and 3D PSC/HC3x-HLCs mono-cultures was found to be toxic for iETV2-ECs, and PSC-Mφs survived poorly when the STEM-Pro hematopoietic culture medium was omitted. Therefore, a medium was created consisting of LDM mixed with StemPro™-34 SFM and addition of all growth factors used for the different PSC-progeny in mono-culture, except for the AAGly supplement.

Similar to the spheroid-like clusters that were formed when single-cell suspensions of HC3X-HLCs were cultured in HepMat hydrogels, single cell suspensions of HC3x-HLCs co-embedded together with PSC-NPCs also led to the spontaneous creation of spheroid-like structures. In addition, in the co-cultures also the appearance of tubular structures can be seen interspersed between the spheroid structures. This apparent spontaneous morphogenesis in both HC3x-HLC mono-culture and the 4-cell co-s occurred during the first 10 days of (co)-culture. The spheroid formation of HC3x-HLCs is apparently not the result of cell proliferation, as expansion of HC3X-hepatoblasts is very limited in 2D culture, even if PSC-HSCs, iETV2-ECs and PSC-Mφs still proliferate beyond the day they were harvested from 2D cultures. Moreover, compared with the input cell ratios (44% HC3x-hepatoblasts, 22% iETV2-ECs, 22% PSC-HSCs, 11% PSC-Mφ) the final composition of the cells present in the 4-cell co-culture was not significantly altered except for an apparent loss of ECs (of the identified cell types, 57% were endodermal, 20% mesenchymal and hematopoietic but only 2% endothelial), also arguing against the fact that cell proliferation in situ underlies the creation of spheroids or tubular structures in mono- and co-cultures. One could argue that the creation of multicellular structures within the hydrogel is in itself responsible for the maturation and further cell fating of HC3X-HLCs as well as NPCs, and that presence of the HepMat hydrogel plays only a minor role. Arguments against this are, first, that removal of a single peptide from the peptide pool resulted in inferior HLC maturation. Second, HLC maturation was highly dependent on different peptide combinations, different stiffness or degradability of the hydrogels, and some hydrogels even decreased maturation of HLCs compared with 2D cultures. Third, although HLC-spheroids could be aggregated by forced aggregation, significantly inferior maturation was observed compared with HLCs matured in HepMat hydrogels.

Although multiplex immunostaining revealed that of all endodermal progeny in HepMat co-cultures, 10% had a phenotype of hepatic progenitors (AFP+/KRT19+) and 7% an intermediary (AFP+/ALB+/CYP3A4−) hepatocyte phenotype, 44% of cells displayed an AFP−/ALB+/CYP3A4+ staining pattern, compatible with mature hepatocytes. In addition, +18.4% of cells co-stained with hepatocyte and mesenchymal markers, suggesting presence of some degree of EMT. 13.4% of cells have a cholangiocyte phenotype, suggesting the creation of biliary ducts from the d8-PSC-hepatoblasts even if such cells were not identified in HC3X-HepMat mono-cultures. Cholangiocyte differentiation from bipotent hepatoblasts is governed by a number of cell-extrinsic signals emanating from mesenchymal structures adjacent to bile ducts. This includes signaling by TGFβ derived from the periportal mesenchyme to commit hepatoblasts to ductal plate cells, and JAGGED1, also expressed in the periportal mesenchyme, which activates the NOTCH2 signalling pathway to support cholangiocyte differentiation. Therefore, the development of biliary ducts in co-cultures containing HSC-like cells but not in mono-cultures of PSC-hepatoblasts may not be surprising. Vascular tube-like structures persisted until at least 32 days in the combinatorial system, which was in stark contrast with PSC-iETV2-EC mono-cultures, consistent with the need for supporting cells for stable vasculogenesis.

The presence of PSC-NPCs supported HLC maturation. As AAGly, crucial for metabolic maturation of HC3x-HLC in 2D culture [Boon et al. cited above] and in HepMat mono-culture, appeared toxic for PSC-iETV2-ECs, the AAGly supplement was removed from the co-culture medium. Nevertheless, even when AAGly was omitted, CYP450 gene expression as well as function reached levels similar to PHHs, demonstrating that the co-culture system in its own supports HLC maturation and metabolic supplementation of the culture medium is no longer required.

In addition, co-culture of PSC-iETV2-ECs with the other cell populations induced LSEC-like gene expression, as a significant further induction of FCGR2B, LYVE1, MRC1 and STAB1 was observed. Even though these marker genes are commonly used to demonstrate presence of LSECs in primary hepatocyte/NPC spheroid cultures, promiscuous expression of scavenging marker genes in KC/Mφs and LSECs exists. Other newly identified LSEC marker genes (such as FCN3, OIT3, CLEC4M, CLEC4G) were not significantly higher in the HepMat co-culture system compared with iETV2-EC HepMat mono-cultures, but this may in part be because the relatively limited number of iETV2-ECs in the final mixture.

In the co-culture also a further decrease in fibrotic HSC markers was seen, including multiple collagen genes, PDGFRalpha the quintessential fibrotic niche associated HSC marker, as well as ACTA2 and LOXL2. Nevertheless, other marker genes such as of RGS5 and IGFBP5, identified by single cell RNA sequencing in candidate non-fibrotic HSCs [Ramachandran et al. cited above], remained low in the HepMat cocultures. PSC-Mφ persisted, even though most putative KC-specific gene markers were not higher in the co-culture system compared to PSC-Mφ-3D mono-cultures.

When the co-cultures were exposed to either TGFβ or OA, a massive increase in pro-fibrogenic and pro-inflammatory genes occurs, compared with any of the 3D mono-cultures, demonstrating that liver inflammation and fibrosis is, as in vivo, dependent on an intricate interaction between HLCs and NPCs. The induction of e.g. IL6 and collagen in the HepMat co-culture appeared to exceed levels reported previously for PSC-HLC-NPC spheroid cultures, possibly due to the higher frequency of NPCs within the HepMat co-culture compared with cultures wherein NPCs were co-differentiated from PSCs. Moreover, the induction of collagens by TGFβ exposure was similar to levels reported for primary liver spheroids consisting of PHHs and the most fibrogenesis-inducing primary liver derived NPCs. Co-culture of HC3x-HLCs with any of the three NPCs separately can be compared, to gain insight in how their combined presence induces this fibro/inflammatory response. The steatosis (evaluated by BODIPY® staining), pro-inflammatory and fibrogenesis inducing effect of OA could be largely blocked by treatment of the culture with OCA, a drug shown in phase III studies to block fibrosis and/or decrease NASH features. By contrast, Elafibranor, which was recently shown to not improve liver fibrosis compared to placebo treatment in a phase III study, did not inhibit OA induced secretion of collagen and IL6 secretion, even if it decreased some of the fibrogenic and inflammatory gene transcript levels, and a decrease in steatosis, albeit somewhat less than with OCA, was seen.

In conclusion, an all-PSC-derived hepatocyte- and NPC-like cell co-culture system was created that significantly improves PSC differentiation and specification to mature hepatic progeny, HSC with a more fibrotic phenotype, and PSC-ECs with a more LSEC phenotype. As the co-culture is based on a fully defined hydrogel composition and well-defined ratios of hepatoblast- and NPC-progenitor cell input, the culture system may be less variable than spontaneous PSC-co-differentiation cultures with or without natural polymer matrices. Finally, the co-culture system is suitable for studying liver steatosis, inflammation and fibrosis as well as assess drugs counteracting these effects.

### Example 1. Combinatorial Screen of Instructive (Responsive) Hydrogels to Identify the Optimal Environment that Sustains Functional Maturation of HLCs

A series of 3D hydrogels was constructed using four-arm-PEG building blocks with functional vinyl sulfone end-groups that could be linked to different adhesion ligand peptides and matrix metalloproteinase (MMP) cleavable cross-linkers. The MMP cleavable peptides, having thiol groups on both ends, as described by Lutolf & Hubbel cited above, were selected based on MMPs present in human liver. These MMPs included DG1 (GCRDVPLSYSGDRCG) [SEQ ID NO:49],

DG2
[SEQ ID NO: 50]
(GCRDGPQGIAGQDRCG),
and
DG3
[SEQ ID NO: 51]
(GCRDGPQGIWGQDRCG),

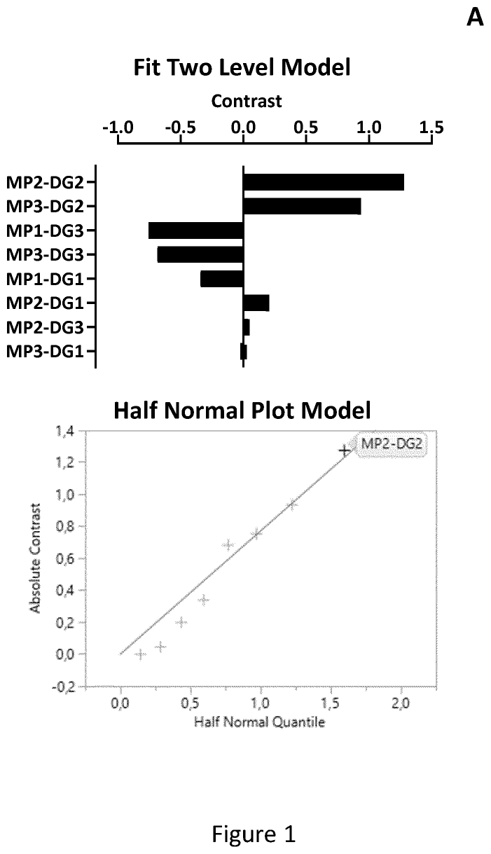

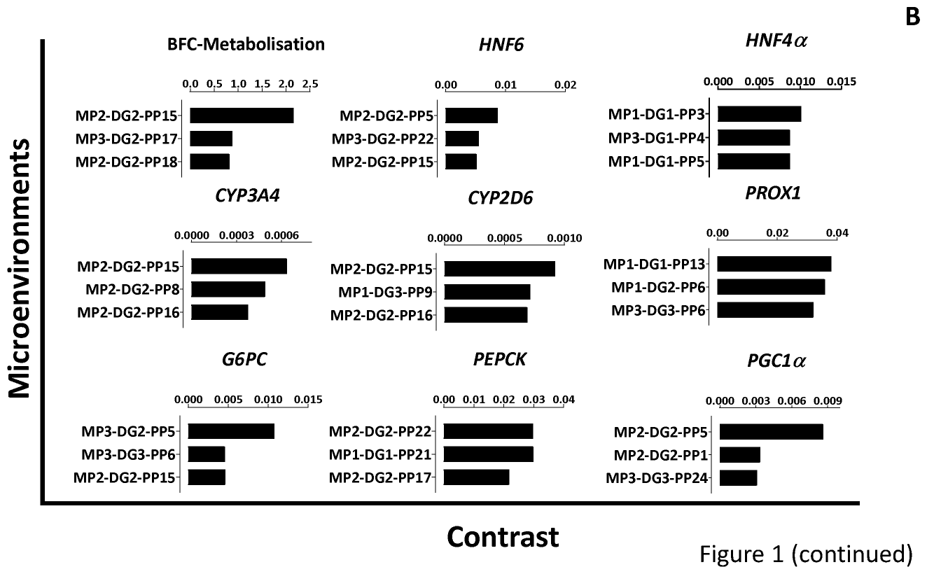

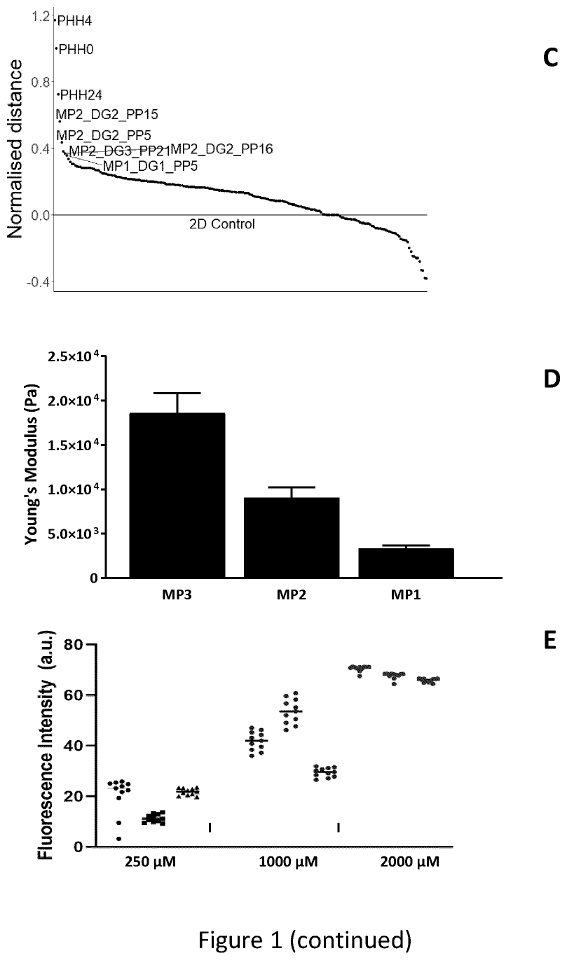

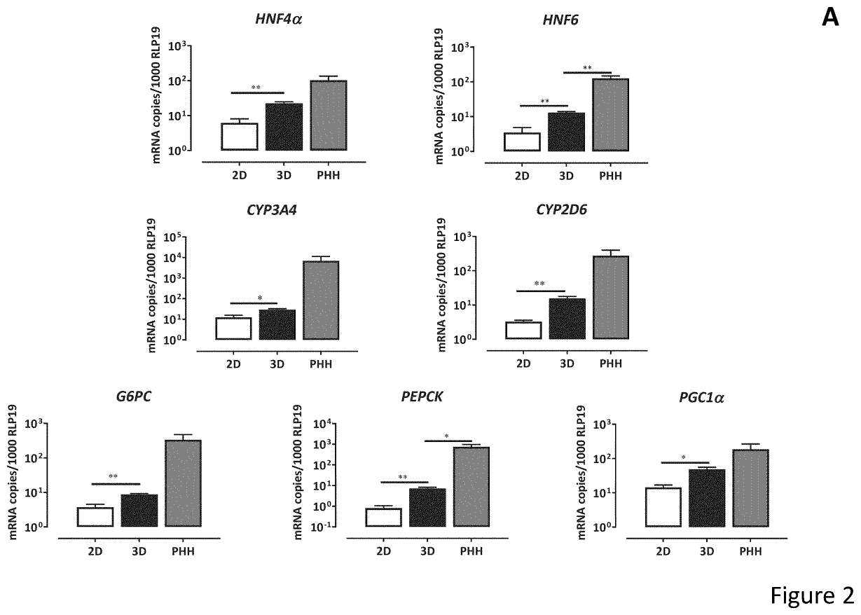

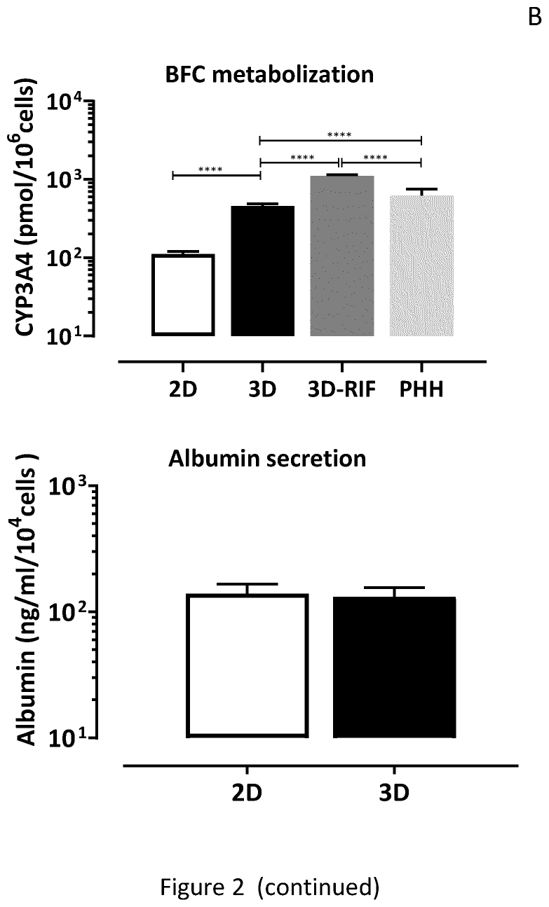

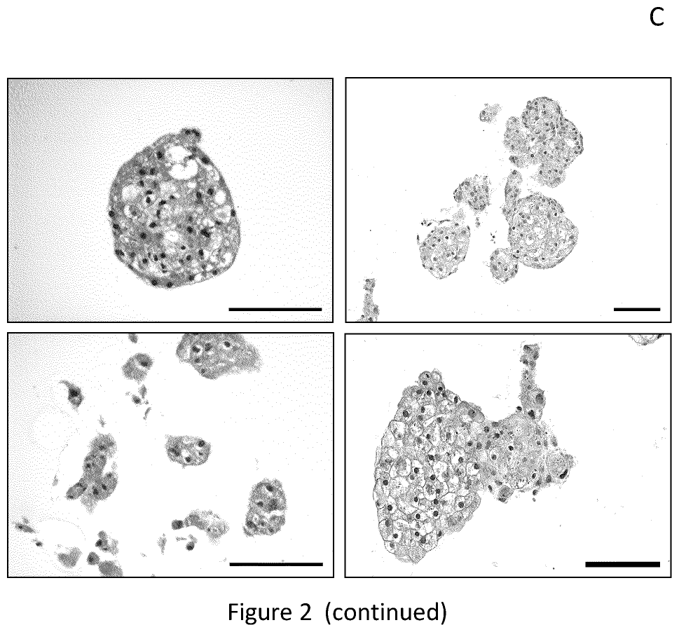

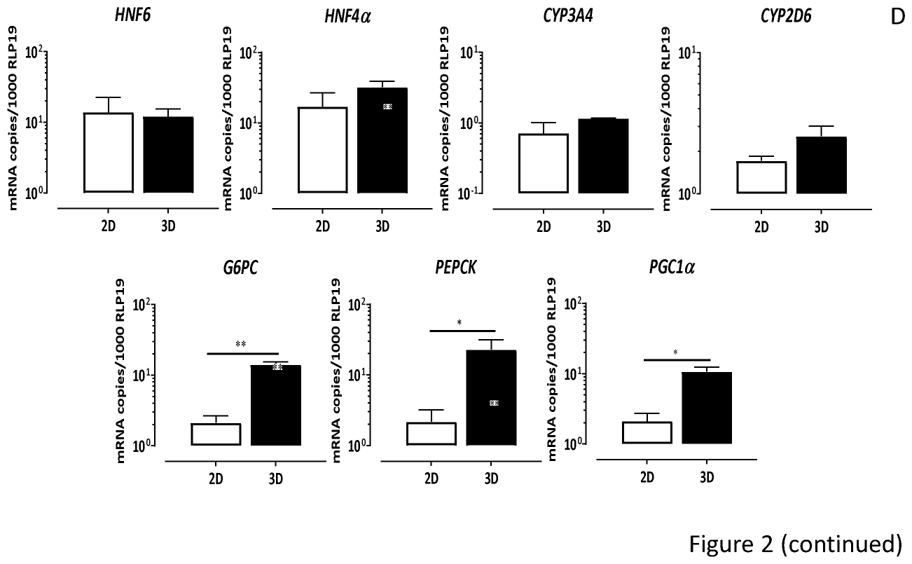

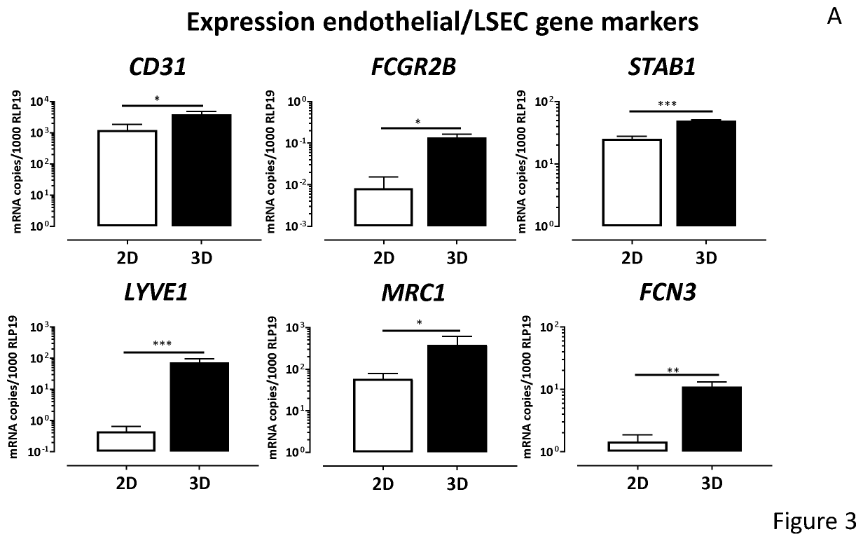

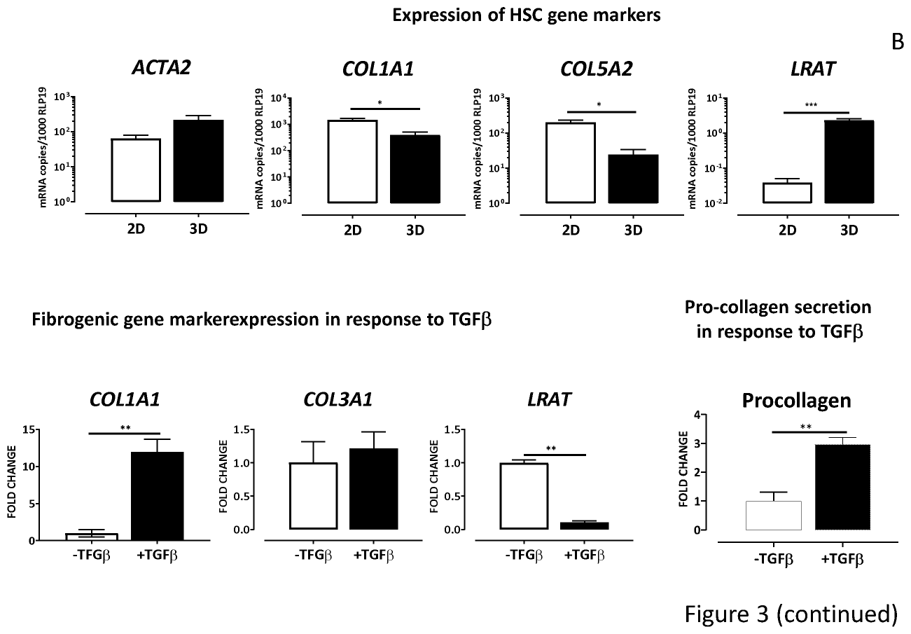

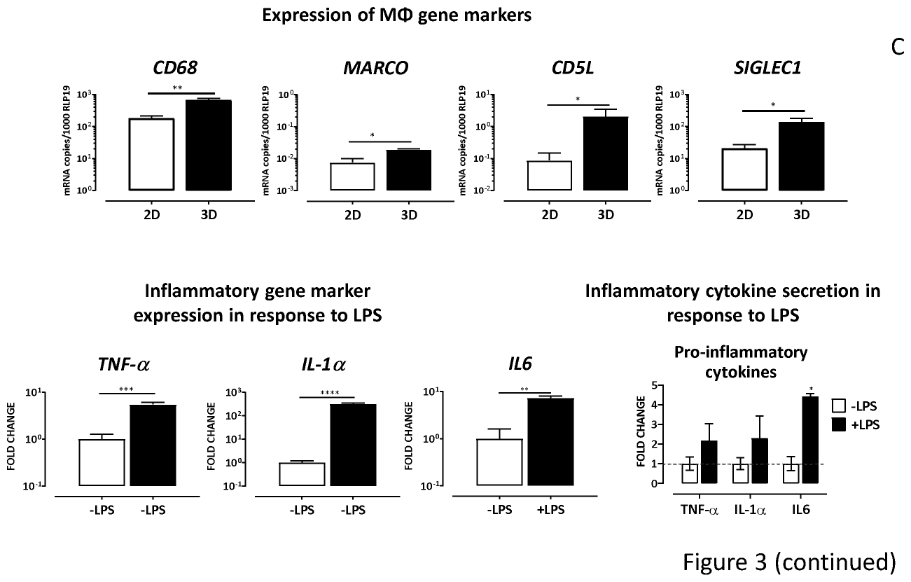

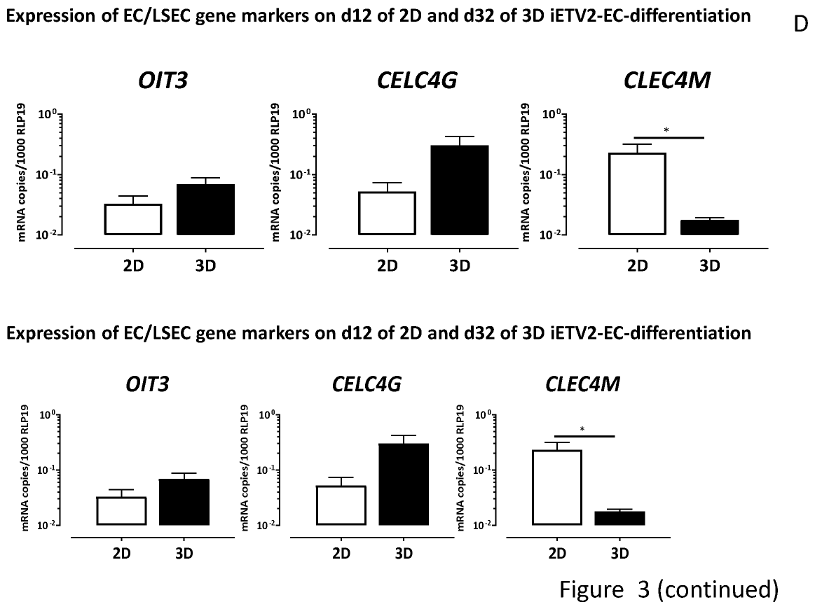

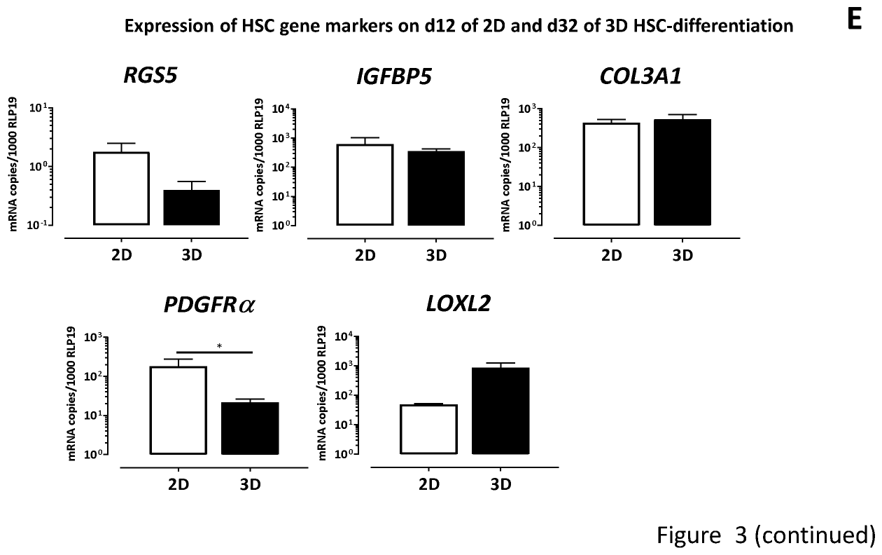

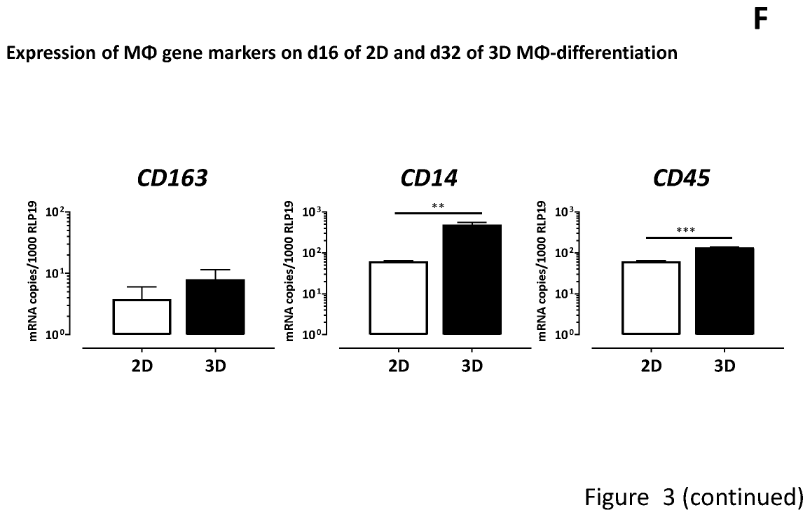

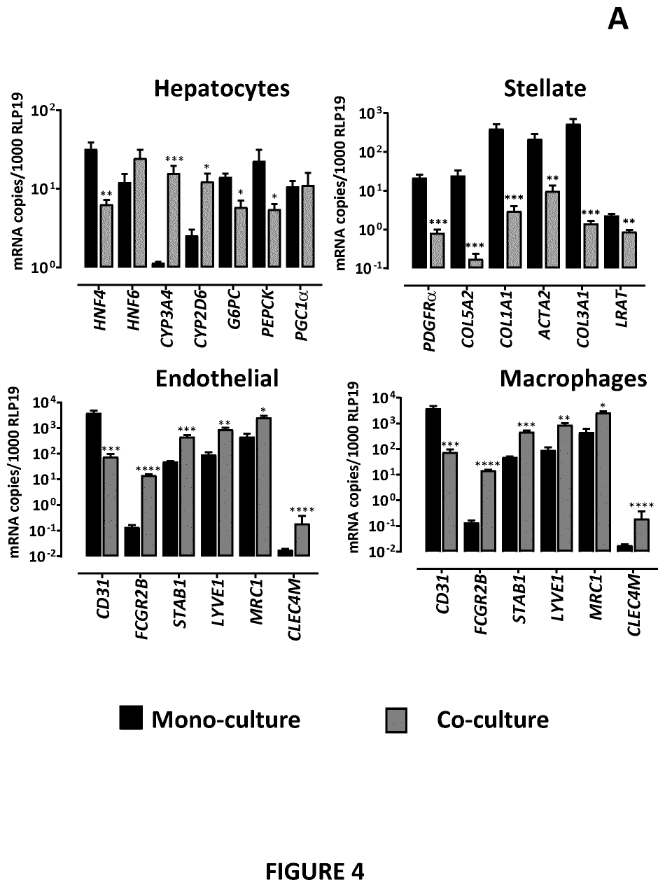

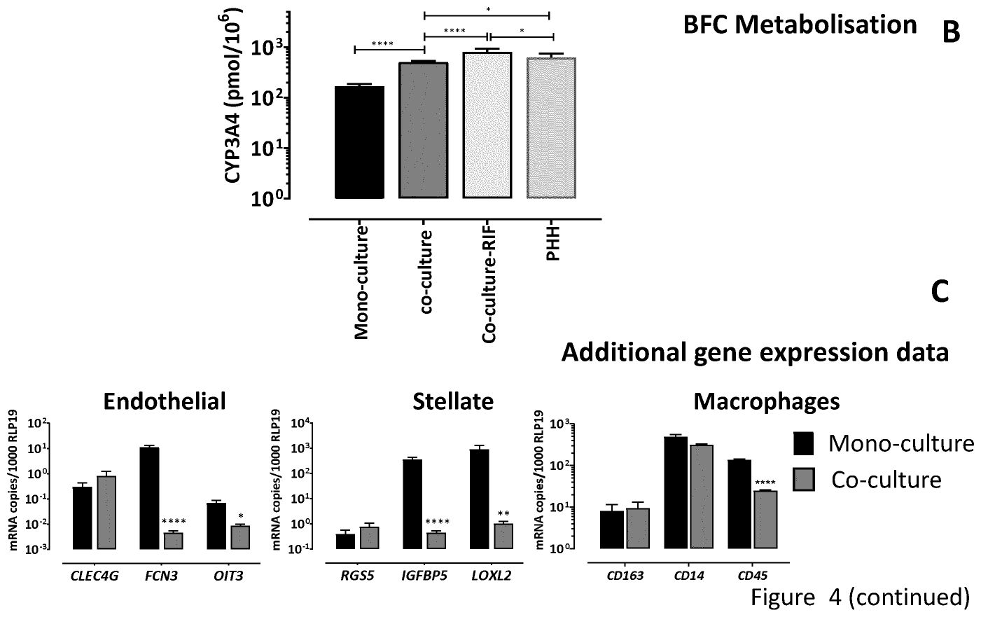

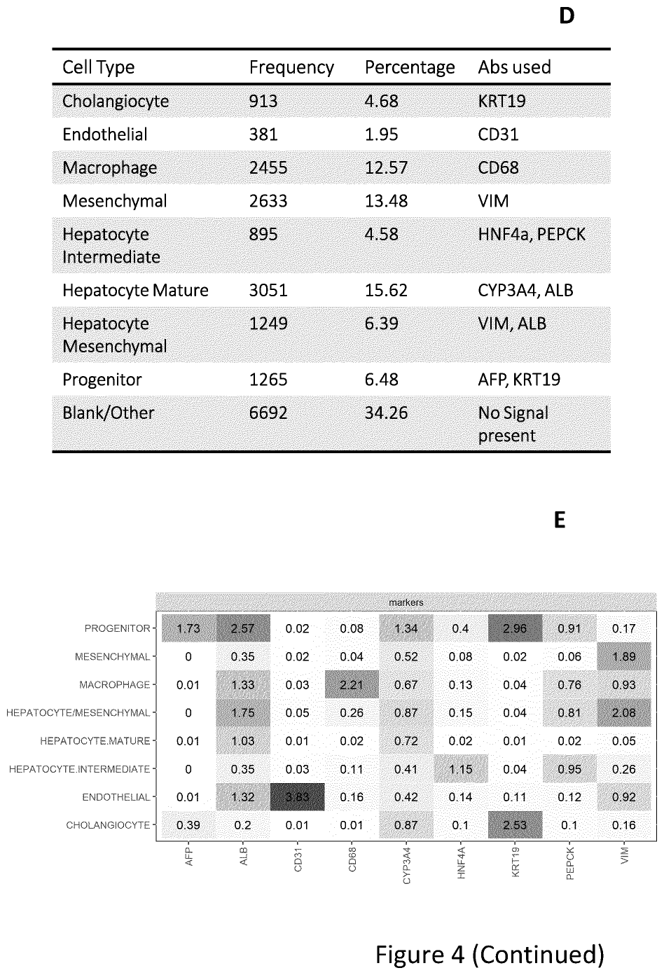

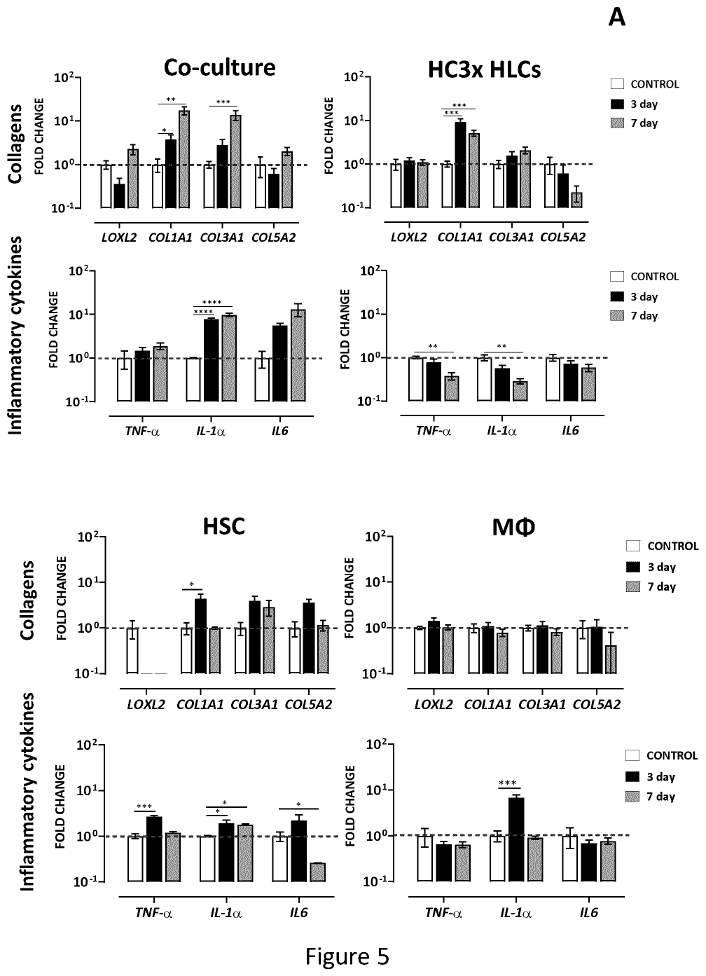

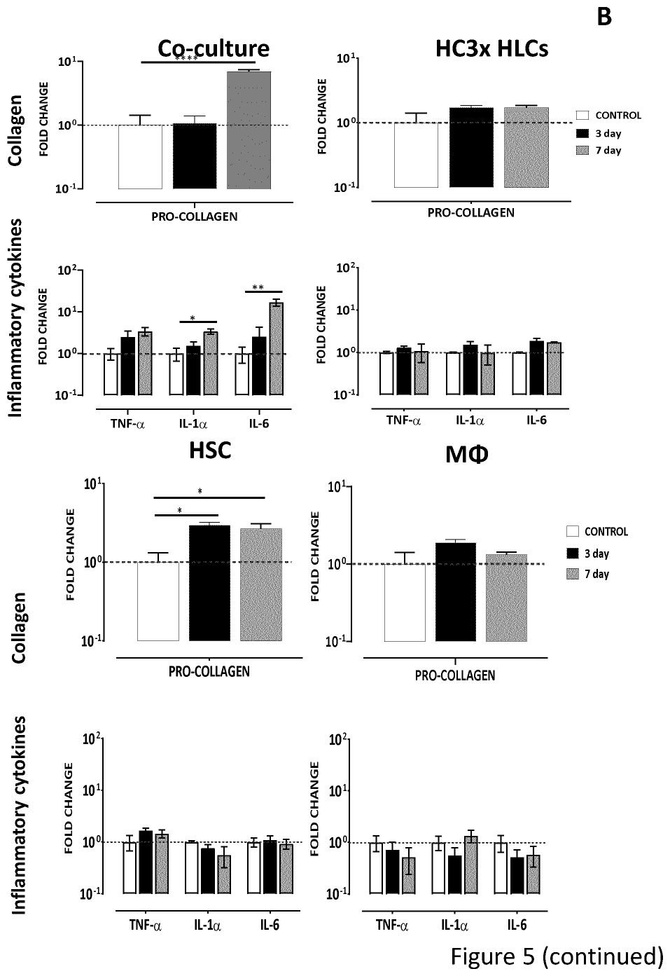

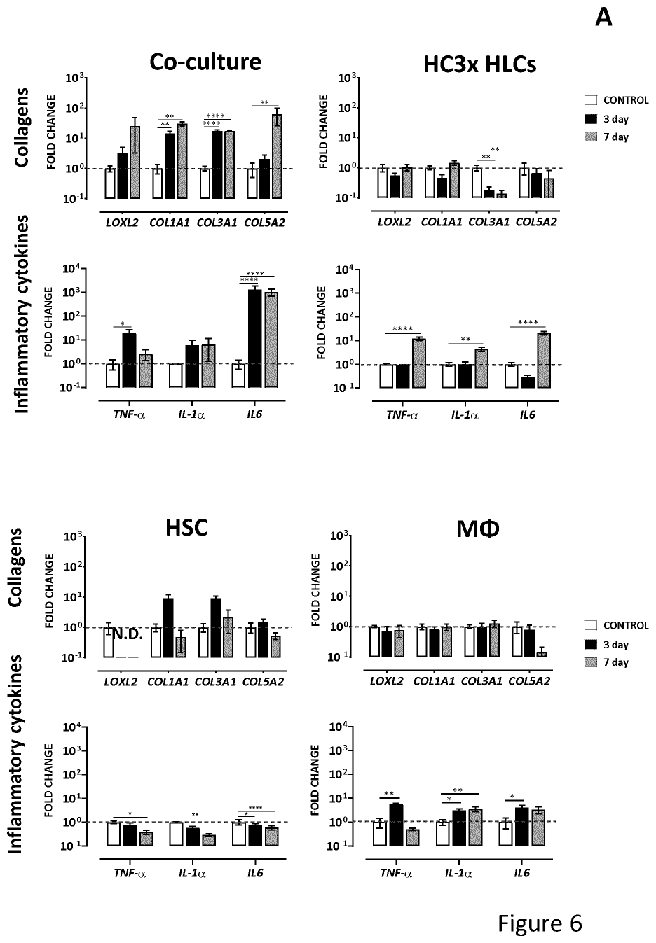

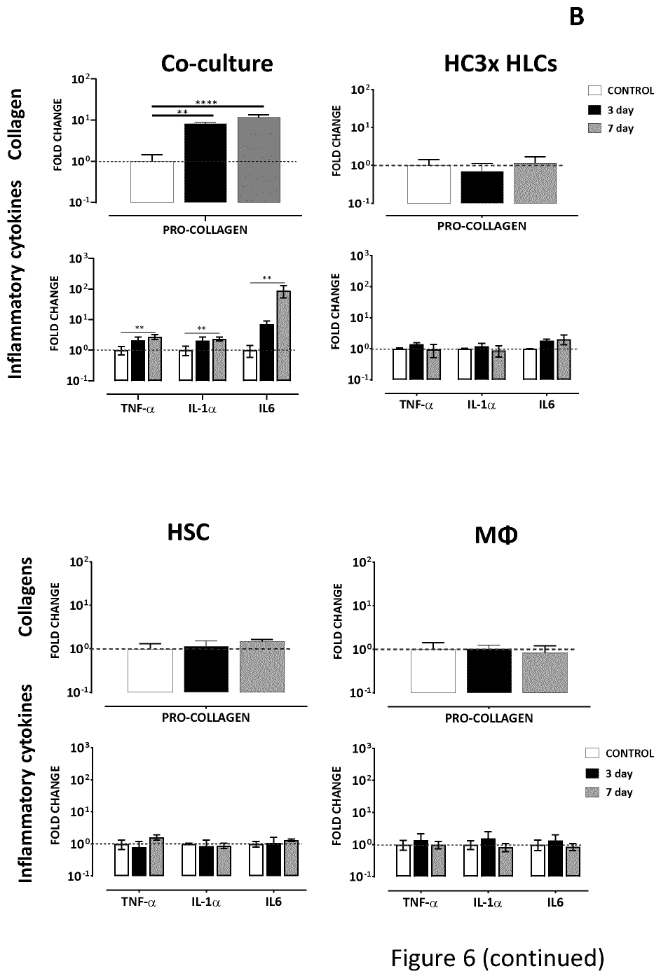

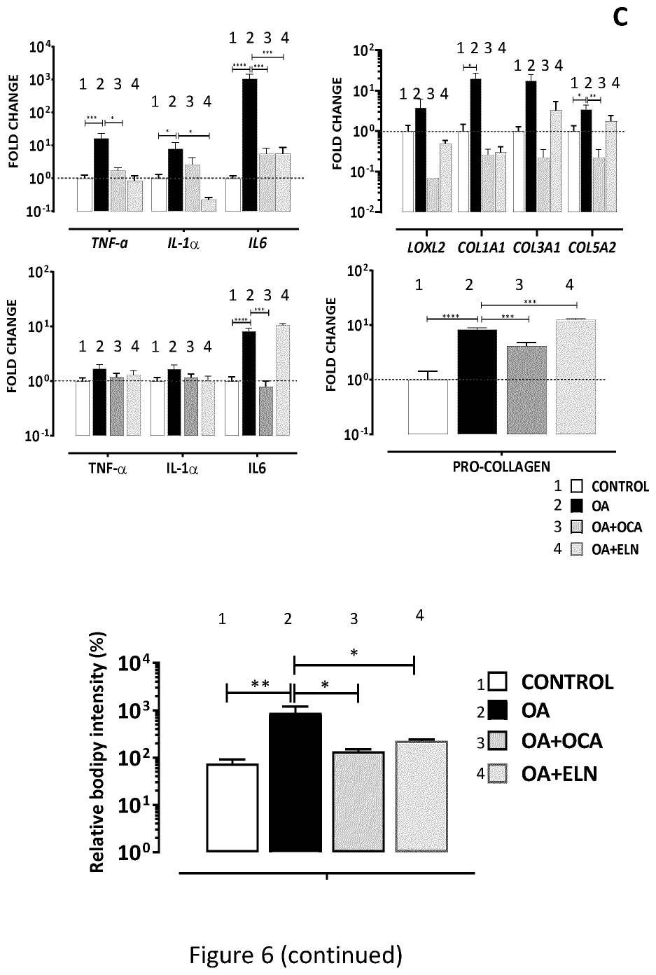

with different degradation kinetics [Patterson et al. cited above]. To vary the stiffness of the hydrogel, three concentrations of PEG polymers were used named MP1 (8% w/v), MP2 (10% w/v) and MP3 (12% w/v). Bulk mechanical properties, measured using a nano-indenter, of the hydrogels containing increasing concentrations of PEG polymers and following peptide functionalisation, were 3 Kpa, 9 Kpa and 20 Kpa, for the MP1, MP2 and MP3-based hydrogels, respectively. For adhesion ligand functionalisation, 24 peptides were selected representing the active part of ECM components (fibronectin, collagen I, collagen III, collagen IV, laminins, perlecan) or cell adhesion molecules (E- and N-cadherin) present in liver (based on data from the human Matrisome project [Naba et al. (2016) Matrix Biol. 49, 10-24] (Table 1). The peptide sequences consisted of Ac-GCGYG-“peptide”-G-NH₂ [SEQ ID NO:53] where “peptide” represented the active component of the entire peptide, and wherein the underlined glycine is optional. Except from peptide P8 in table 1, wherein an additional DPG sequence is present. To demonstrate the efficient binding of the ECM/CAM peptides to the PEG backbone, fluorescently labelled peptides were conjugated to PEG hydrogels, and demonstrated a concentration-dependent increase in fluorescence intensity (FIG. 1E).

TABLE 1
peptide description

| | | Modified peptide | | | |
|---|---|---|---|---|---|
| Code | Peptide sequence | sequence | Origin | Domain | Receptor |
| P1 | RGD | GCGYG RGD G | FN | III-10 | α5ß1 |
| | | [SEQ ID NO: 25] | | | |
| P2 | RGDS | GCGYG RGDS G | FN | III-10 | αIIbß3 |
| | [SEQ ID NO: 2] | [SEQ ID NO: 26] | | | |
| P3 | KQAGDV | CGGYG KQAGDV G | FN | | αIIbß3 |
| | [SEQ ID NO: 3] | [SEQ ID NO: 27] | | | |
| P4 | REDV | GCGYG REDV G | FN | IIICS-5 | α4ß1 |
| | [SEQ ID NO: 4] | [SEQ ID NO: 28] | | | |
| P5 | PHSRN | GCGYG PHSRN G | FN | III-9 | α5ß1 |
| | [SEQ ID NO: 5] | [SEQ ID NO: 29] | | | |
| P6 | KRLDGS | GCGYG KRLDGS G | FN | γ chain | αvß2 or |
| | [SEQ ID NO: 6] | [SEQ ID NO: 30] | | | αMß2 |
| P7 | KGHRGF | GCGYG KGHRGF G | COL-I | α1 | Heparin |
| | [SEQ ID NO: 7] | [SEQ ID NO: 31] | | | |
| P8 | YIGSR | GCGYG DPG YIGSR | LAM | ß1 Chain- | α1ß1, |
| | [SEQ ID NO: 8] | G | | III | α3ß1 |
| | | [SEQ ID NO: 32] | | | |
| P9 | IKVAV | GCGYG IKVAV G | LAM | α1 | αvß3 |
| | [SEQ ID NO: 9] | [SEQ ID NO: 33] | | | |
| P10 | IKLLI | GCGYG IKLLI G | LAM | α1 (L-111) | α3ß1, |
| | [SEQ ID NO: 10] | [SEQ ID NO: 34] | | | heparin |
| P11 | DGEA | GCGYG DGEA G | COL-I | α1 | α2ß1, |
| | [SEQ ID NO: 11] | [SEQ ID NO: 35] | | | GPIV |
| P12 | GTPGPQGIAGQRDV | GCGYG | COL-I | α1 | Not |
| | V | TPGPQGIAGQRDVV | | | known |
| | [SEQ ID NO: 12] | G | | | |
| | | [SEQ ID NO: 36] | | | |
| P13 | GFOGER | GCGYG FOGER G | COL-I | α1 | α1ß1, |
| | [SEQ ID NO: 13] | O is hydroxyproline | | | α2ß1, |
| | O is hydroxyproline | [SEQ ID NO: 37] | | | α11ß1 |
| P14 | GLOGEN | GCGYG LOGEN G | COL-III | α1 | α1ß1 |
| | [SEQ ID NO: 14] | [SEQ ID NO: 38] | | | |
| | O is hydroxyproline | O is hydroxyproline | | | |
| P15 | GLKGEN | GCGYG LKGEN G | COL-III | α1 | α2ß1 |
| | [SEQ ID NO: 15] | [SEQ ID NO: 39] | | | |
| P16 | TAGSCLRKFSTM | GCGYG | COL-IV | α1 | α2ß1, |
| | [SEQ ID NO: 16] | TAGSCLRKFSTM G | | | Heparin |
| | | [SEQ ID NO: 40] | | | |
| P17 | FYFDLR | GCGYG FYFDLR G | COL-IV | α1 | α1ß1, |
| | [SEQ ID NO: 17] | [SEQ ID NO: 41] | | | α2ß1 |
| P18 | TWSKVGGHLRPGIV | CGGYG | PERLECAN | Perlecan IV | IgB |
| | QSG | TWSKVGGHLRPGIVQ | | | |
| | [SEQ ID NO: 18] | SG G | | | |
| | | [SEQ ID NO: 42] | | | |
| P19 | VAPG | GCGYG VAPG G | ELASTIN | Elastin | Elastin |
| | [SEQ ID NO: 19] | [SEQ ID NO: 43] | | | receptor |
| P20 | VAEIDGIEL | GCGYG VAEIDGIEL G | TENACIN-C | Tenascin-c | α9ß1 |
| | [SEQ ID NO: 20] | [SEQ ID NO: 44] | | | |
| P21 | LFSHAVSSNG | GCGYG | E- | | EC1 |
| | [SEQ ID NO: 21] | LFSHAVSSNG G | CADHERIN | | domain |
| | | [SEQ ID NO: 45] | | | |
| P22 | HAVDI | GCGYG HAVDI G | N- | | EC1 |
| | [SEQ ID NO: 22] | [SEQ ID NO: 46] | CADHERIN | | domain |
| P23 | ADTPPV | GCGYG ADTPPV G | E- | | EC1 |
| | [SEQ ID NO: 23] | [SEQ ID NO: 47] | CADHERIN | | domain |
| P24 | LRRFSTMPFMFCNI | GCGYG | COL-IV | α5 | αvß3, |
| | NNVCNF | LRRFSTMPFMFCNIN | | | αvß1 |
| | [SEQ ID NO: 24] | NVCNF G | | | |
| | | [SEQ ID NO: 48] | | | |

To define the environment that most optimally sustained PSC-HLCs and to avoid prohibitively exhaustive screening, a discrete numeric design was used (using JMP pro (SAS)) to create a design-of-experiment (DOE). This resulted in a total of 216 microenvironments, each consisting of one of three stiffnesses (MP1-3), one of three MMP linkers (DG1-3), and pools of 6 peptides (PP1-PP24) (Table 2).

TABLE 2
Peptide pools included in the DOE Peptide combinations
using JMP-discrete numeric design

| | |
|---|---|
| Peptide pool | peptides |

| | | | | | | |
|---|---|---|---|---|---|---|
| PP1 | P4 | P9 | P11 | P13 | P18 | P19 |
| PP2 | P5 | P7 | P11 | P15 | P16 | P24 |
| PP3 | P4 | P7 | P11 | P13 | P17 | P21 |
| PP4 | P3 | P8 | P11 | P14 | P17 | P24 |
| PP5 | P6 | P8 | P11 | P13 | P16 | P20 |
| PP6 | P5 | P8 | P12 | p13 | p16 | P21 |
| PP7 | P1 | P10 | P12 | P14 | P18 | P24 |
| PP8 | P6 | P10 | P11 | p13 | p16 | P21 |
| PP9 | P3 | P7 | P12 | P13 | P18 | P22 |
| PP10 | P2 | P9 | P11 | P15 | P18 | P20 |
| PP11 | P1 | P10 | P11 | P13 | P18 | P22 |
| PP12 | P5 | P9 | P11 | P14 | P17 | P22 |
| PP13 | P6 | P7 | P12 | p13 | P18 | P19 |
| PP14 | P4 | P10 | P12 | P14 | P17 | P20 |
| PP15 | P5 | P8 | P12 | P13 | P16 | P23 |
| PP16 | P1 | P7 | P11 | P14 | P16 | P23 |
| PP17 | P3 | P9 | P12 | P14 | P16 | P21 |
| PP18 | P2 | P9 | P12 | P13 | P16 | P24 |
| PP19 | P2 | P7 | P12 | P15 | P17 | P20 |
| PP20 | P4 | P10 | P12 | P15 | P16 | P22 |
| PP21 | P3 | P10 | P11 | p13 | P16 | P19 |
| PP22 | P2 | P8 | P11 | P14 | P18 | P23 |
| PP23 | P6 | P9 | P12 | P15 | P17 | P23 |
| PP24 | P1 | P8 | P12 | P13 | P17 | P19 |

To test the effect of these microenvironments on HLC maturation, PSC-progeny differentiated to the hepatic lineage for 8 days in 2D culture (hepatoblast stage) were seeded at 3×10⁵ cells/10 μL of the different hydrogel compositions, and allowed the cells to mature until day 20. Maturation was assessed by a combination of RT-qPCR for mature hepatocyte marker genes (Table 3) and benzyloxy-4-trifluoromethylcoumarin (BFC) metabolisation (function of CYP3A4 (and CYP1A2)). All data was compared with HLCs maintained until day 20 in 2D culture, and PHHs (freshly thawed [PHH0]; cultured for 4 h [PHH4], and 24 h [PHH24]).

TABLE 3
markers genes used for qPCR

| Intended target | Gene |
|---|---|
| Reference gene | RPL19 |
| Endothelial/LSEC | PECAM1, CLEC4G, CLEC4M, FCGR2B, FCN3, LYVE1, MRC1, |
| markers | OIT3, STAB1 |
| Hepatocyte | AAT, AFP, ALB, HNF4α, HNF6, HNF1a, NTCP, MRP2, |
| markers | NFKBIa, CYP3A4, CYP2D6, PXR, GSTμ, GSTpi, Prox1, |
| | G6PC, PEPCK, CEPBA, CEBPB, PGC1, KAT2A, SIRT1, |
| | ATP5A1, GCK, CPT1a, TFAM, CYTC, MPC1, MPC2, NR5A2, |
| | ApoE, ApoA1, PFKB3, Acox1, CTGF, ANKRD1, YAP1, TAZ, |
| | ALAS1 |
| Hepatic stellate | COL5A2, LOXL2, COL1A1, COL3A1, RGS5, PDGFα, IGFBP5, |
| cell markers | ADAMTS1, ALCAM, ACTA2, LRAT |
| Macrophage/Kupffer | MARCO, CD163, CD5L, SEGLEC1, CD68, CD14, CD45 |
| cell markers | |
| Inflammatory | IL-1a, IL-6, TNF-α |
| cytokines | |

Using the Fit-Two-Level model screening module, which identifies effects that have a large impact on the response based on the sparsity-of-effects principle, it was found that among gels with different mechanical properties (i.e. the combinatorial effect of DG1, DG2 or DG3 with MP1, MP2 or MP3) the combination of MP2 and DG2 yielded hepatic progeny with the highest BFC metabolisation. This was confirmed using a half-normal probability plot, which estimates, via a least-squares estimation, the effect of a given main effect or interaction and its rank relative to other main effects and interactions, enabling the ranking of factors by importance (FIG. 1A). The combined effect of mechanical properties and functionalisation by different peptide pools on hepatocyte maturation was analysed, using BFC metabolisation and transcript levels of eight hepatic marker genes (HNF4a, HNF6, PROX1, CYP3A4, CYP2D6, PEPCK, G6PC, PGC1a) as readout. The Fit-Two-Model module identified that MP2-DG2 hydrogels functionalised with peptide pool 15 (PP15) ranked among the top 3 microenvironments for five out of nine 5/9 markers tested (FIG. 11B). Calculation of normalised Manhattan distances (based on log 2 fold changes measured by RT-qPCR) revealed that hepatic progeny from the MP2-DG2-PP15 hydrogel-based cultures ranked the closest to PHHs. (FIG. 1C). Finally, hierarchical clustering based on 39 different mature hepatocyte gene marker transcripts also demonstrated that the hepatic progeny from MP2-DG2-PP15 hydrogel-based cultures clustered most closely to PHHs.

To confirm the optimal peptide pool for HLC maturation, the screen was repeated only using the MP2-DG2 backbone and functionalised by any of the 24 different peptide pools. Hierarchical clustering analysis of RT-qPCR results from day 20 progeny identified again the PP15 peptide pool based functionalised MP2-DG2 hydrogel to be the most supportive for HLC maturation. The peptide combination PP15, containing the fibronectin peptide P5, the laminin peptide P8, the collagen I peptides P12 and P13, the collagen IV peptide P16 and the E-cadherin peptide P23, in 10% PEG-hydrogels cross-linked with the

[SEQ ID NO: 50]
GCRDGPQGIAGQDRCG

MMP degradable linker, generates PSC-HLCs that cluster significantly closer to PHHs than 2D cultured PSC-HLCs, determined both transcriptionally and functionally. As two different collagen I peptides were in this specific 6-peptide combination, it was tested if both were required. However, removal of either of the 2 peptides significantly decreased transcript levels of CYP3A4 and CYP2D6. Hence for all subsequent studies this 6-peptide combination embodiment was retained. The MP2-DG2-PP15 hydrogel composition was termed “hepatocyte-maturation” or “HepMat” hydrogel.

### Example 2. The Hepatocyte Maturation (HepMat) Hydrogel Supports Further Maturation of HLCs Generated from Genome Engineered PSC (HC3x-PSC) and Cultured

Boon et al. cited above, demonstrated that PSC-hepatic differentiation is significantly enhanced when PSCs are genetically engineered to inducibly overexpress three transcription factors (HNF1A, FOXA3 and PROX1, termed HC3x-PSC) from day 4 onwards, and are cultured in amino acid (AA)-enriched medium (liver differentiation medium [LDM] supplemented with 16 ml non-essential AA solution, 8 ml of essential AA solution per 100 ml of LDM, and 20 g/I glycine; termed AAGly medium). It was first determined if the PP15-functionalised MP2-DG2 hydrogel also is the most optimal hydrogel composition to induce maturation of HLCs when cultured in AAGly medium. among the different peptide pool combinations, the MP2-DG2-PP15 (HepMat) hydrogel cultured HLCs again clustered most closely to PHH. As the Boon et al also demonstrated significant further improvement in HLC maturation, it was next addressed if HC3x genome engineered HLCs maintained for 40 rather than 20 days in AAGly medium, cultured in in HepMat hydrogels would attain even further maturation beyond culture in 2D. RT-qPCR analysis demonstrated that HC3x progeny 32 days after embedding in HepMat hydrogels and maintained in AAGly medium expressed significantly increased transcript levels for all genes tested (HNF4a, HNF6, CYP3A4, CYP2D6, PEPCK, G6PC and PGC1a) compared with continuous culture in 2D (FIG. 2A). Moreover, the activity of CYP3A4 of HC3x-HLCs, already high in cells matured in 2D-AAGly-HC3x-HLCs [Boon et al. cited above] (FIG. 2B), increased significantly when cells were cultured in 3D-HepMat-AAGly hydrogels (FIG. 2B). BFC metabolisation of 3D-HepMat-AAGly HC3x-HLCs was significantly induced by treatment with rifampicin. However, no significant differences were observed in albumin secretion level between 2D-AAGly and 3D-HepMat-AAGly cultured cells. Interestingly, although a significant increase in transcript levels for G6PC and PGC1a was also seen when HC3x cells were cultured for in 3D-HepMat-LDM between day 12 and 40 compared with 40 days in 2D LDM-culture, the cytochrome transcript levels were not induced. Thus in line with Boon et al. cited above, AA levels in the culture medium are important for hepatocyte maturation also in 3D-HepMat cultures. Histological analysis of the HC3x progeny cultured in 3D HepMat-AAGly cultures identified polygonal cells with an eosinophilic and clear/vacuolated cytoplasm, with a round shaped nucleus, and present in a cohesive pattern, highly reminiscent of hepatocytes (FIG. 2C). Immunostaining and Fluorescence microscopy imaging confirmed the hepatocyte identity of the cells (nearly 100% albumin and HNF4a positive). Also CYP3A4 and PEPCK positive cells could be identified. There was little overlap between CYP3A4 and PEPCK staining, consistent with hepatocyte diversity in primary human liver. In addition, most cells had cell surface staining of NTCP, and expression of MRP2 both in the cell cytoplasm and more punctate on the cell membrane.

### Example 3. The Hepatocyte Maturation Hydrogel Maintains PSC-Derived Non-Parenchymal Cells (NPCs)

As hepatocytes are located in close proximity with NPCs in liver sinusoids in vivo, the HepMat hydrogel that supports HLC progeny should also support maintenance (and maturation) of PSC-derived NPCs. PSC-ECs, PSC-HSCs and PSC-Mφs harvested from 2D cultures in their respective culture media, were embedded in HepMat hydrogel, maintained the 3D cultures for 32 days and assessed the survival and phenotype by RT-qPCR, immunostaining or flow cytometry, and functional studies.

To generate ECs, PSCs were used wherein the master-regulator, ETV2, was incorporated in the safe harbor locus, AAVS1, under the control of a TET-ON promoter (iETV2-PSC). The ETV2 transcription factor was induced with doxycycline from day 0 of differentiation. By flow cytometry, iETV2-PSC progeny on day 8 was nearly 100% CD31 and KDR double positive (FIG. 3D). RT-qPCR demonstrated that iETV2-PSC progeny expressed high levels of CD31, but not transcripts for LSEC-specific genes (FGFR2B, STAB1, and CLEC4G; and the more recently defined LSEC markers FCN3, OIT3, CLEC4M [Ramachandran et al. cited above] (FIGS. 3A and 3D). 3×10⁵ iETV2-ECs harvested on day 12 from 2D cultures were embedded in 10 μl HepMat hydrogel. iETV2-ECs survived for 32 days (calcein-AM staining), and started to form tubes 5-6 days after embedding in the gels. However, these tubes disintegrated within 2-3 days. When the medium was supplemented with endothelial cell growth supplement (ECGS), tube formation in the HepMat hydrogel was stable for ±2 weeks (FIG. 3A). RT-qPCR 32 days after embedding iETV2-ECs in the HepMat hydrogel demonstrated persistent expression of CD31, and a modest induction of some (FCGR2B, STAB1, LYVE1 and FCN3) but not all (OIT3, MRC1, CLEC4G and CLEC4M) genes considered to be LSEC specific compared with iETV2-ECs harvested on d12 from 2D cultures (FIG. 3A). Nearly 100% of d32 HepMat-cultured iETV2-ECs expressed VE-Cadherin (FIG. 3A). Thus, iETV2-ECs could be maintained in the HepMat hydrogel for at least 32 days, with stable tube formation for ±2 weeks.

PSCs were differentiated towards HSCs based on a protocol described in Coll et al. (2018) Cell Stem Cell. 23(1), 101-13 e7.

Day 12 HSC expressed ACTA2, PDG1FRα, COL1A1, COL3A1, COL5A2 and LOXL2 but low levels of LRAT (important for Vitamin-A metabolism in quiescent HSCs) and the more recently identified non-fibrotic HSC marker genes, RGS5 and IGFBP5 (FIGS. 3B and 3E). Accutase-harvested d12 PSC-HSCs were embedded at 3×10⁵ cells/10 μl HepMat hydrogel, and maintained for 32 days in HSC medium. HSCs survived for 32 days in the HepMat hydrogel (Calcein-AM staining; FIG. 3B). RT-qPCR on day 32 demonstrated a significant decrease in expression levels of COL3A1 and COL5A2, and a significant increase in LRAT expression compared with HSCs harvested from 2D cultures, even if expression levels of other fibrotic (COL1A1, ACTA2, LOXL2) and non-fibrotic (RGS5 and IGFBP5) HSC gene markers remained unchanged (FIGS. 3B and 3E). This transcriptional pattern suggests that culture of PSC-HSCs in 3D may induce at least a partial deactivation of HSCs. Consistently, HSCs cultured in HepMat hydrogels stored retinol, as shown by BODIPY® staining and presence of typical blue autofluorescence elicited by UV light, even when cultured with low concentrations of palmitic acid (FIG. 3B). To demonstrate functionality of the HepMat-embedded HSCs, cultures on d32 were treated with 25 ng/mL TGFβ for an additional 24 hours. RT-qPCR demonstrated a significant increase in expression of COL1A1 (but not LOXL2) and a significant decrease in LRAT transcript levels (FIG. 3B). TGFβ also induced significantly higher levels of secreted levels of pro-collagen (FIG. 3B). Thus, PSC-HSCs can be maintained for at least 32 days in the HepMat hydrogels. Moreover, culture of HSCs in these hydrogels may at least in part induce a less fibrotic HSC phenotype while still allowing TGFβ-mediated activation.

Mφs were generated from PSCs, using a protocol adapted from van Wilgenburg et al. (2013) PLoS One. 8, e71098 (FIG. 3C). Day 16 PSC-Mφs expressed typical Mps genes, including CD163 and CD45 but low levels of putative marker genes for liver specific Mps (also termed Kupffer cells (KCs), such as MARCO, CD5L and SIGLEC1 [Ramachandran et al. cited above]. Day 16-PSC-Mφs were then embedded at 3×10⁵ cells in 10 μL HepMat hydrogel, and maintained in macrophage medium for 32 days. PSC-Mφs survived for 32 days in the HepMat hydrogels (Calcein-AM staining and FACS FIG. 3C). FACS also demonstrated maintenance of CD45 expression on nearly 100% of cells (FIG. 3C). RT-qPCR demonstrated that typical Mφ marker genes remained expressed. An increase in CD68, MARCO, CD5L transcript levels, genes were found to be associated with a KC phenotype [Ramachandran et al. cited aboved](FIG. 3C). To demonstrate functionality of the PSC-Mφs after 32 days in HepMat cultures, the cultures were treated with 100 ng/ml lipopolysaccharide (LPS) for an additional 48 hours. RT-qPCR demonstrated that LPS significantly induced expression of TNFα, IL1α and IL6 (FIG. 3C) and an increased secretion of all three cytokines, which was significant for IL6 (FIG. 3C). Thus, PSC-Mφs could also be functionally maintained for at least 32 days in HepMat hydrogels.

### Example 4. Co-Culture of HC3x-HLCs, iETV2-ECs, PSC-HSCs and PSC-Mφs in Hepatocyte Maturation (HepMat) Hydrogels

As the HepMat hydrogel, optimised to support HC3x-HLC progeny, also supported iETV2-ECs, PSC-HSCs and PSC-Mφs, it was tested if the hydrogel would support co-culture of the four cell types, and if this would enhance the maturation/function of the different cells. Different cell ratios and the medium composition were optimised. A ratio of HC3x-HLCs:iETV2-ECs:PSC-HSCs:PSC-Mφs of 2:1:1:0.5 was optimal to retain all 4 cell types until d32 of co-culture (total number embedded 3×10⁵ cell/10 μL). Supplementation of medium with AAGly, although beneficial for HC3x-HLCs maturation (FIG. 2D), was toxic for iETV2-ECs. Hence, the AAGly supplement was removed from the co-culture medium. As the presence of StemPro™-34 SFM medium appeared necessary for PSC-Mφ maintenance, the co-culture medium consisted of a 1:1 mix of LDM and StemPro™-34 SFM. Finally, all soluble ligands used in mono-culture were incorporated in the final co-culture medium as indicated in table 4.

TABLE 4
Growth factors/cytokines/additives for co-culture differentiation

| | Cytokines | | |
|---|---|---|---|
| Medium | Day 0-4 | Day 4-8 | Day 8-32 |
| LDM + | aFGF (20 ng/mL) | HGF (20 ng/mL) | HGF (20 ng/mL) |
| Retinol + | bFGF (10 ng/ml) | bFGF (10 ng/ml) | bFGF (10 ng/ml) |
| Palmatic | VEGF (50 ng/mL) | VEGF (50 ng/mL) | VEGF (50 ng/mL) |
| acid + | ECGS (20 μl/ml) | ECGS (20 μl/ml) | ECGS (20 μl/ml) |
| StemPro ™- | MCSF(50 ng/mL) | MCSF(50 ng/mL) | MCSF(5 ng/mL) |
| 34 SFM | FLT3(50 ng/mL) | FLT3(50 ng/mL) | 2% Fetal |
| | GM-CSF (25 ng/mL) | GM-CSF (25 ng/mL) | bovine serum |
| | 2% Fetal | 2% Fetal | |
| | bovine serum | bovine serum | |

To characterise the co-cultures, marker gene expression was analysed for the different cell populations. It should be kept in mind that this approach is compromised by the fact that the housekeeping gene transcript level is derived from all four cell types, and that some transcripts can be present in two or more cell populations. As the culture medium was no longer supplemented with the AAGly cocktail, hepatic gene expression levels in d32 HepMat co-cultures were compared with d32 HC3x-HepMat mono-cultures maintained in LDM without AAGly supplementation (FIG. 2B). Transcripts for CYP3A4 and CYP2D6, were significantly higher in HepMat co-cultures than HC3x-HepMat mono-cultures (FIG. 4A). Consistently, BFC metabolisation was significantly higher in HepMat co-cultures than in 3D-HepMat-HC3x mono-cultures, reaching levels similar to suspension cultured PHHs (FIG. 4B). Again, CYP3A4 activity could be induced by Rifampicin. This therefore strongly suggests that co-culture of NPCs with HC3x-HLCs in induce significant metabolic maturation of HC3x-HLCs, and this independent of AAGly required to induce maturation in 2D or 3D HLC-mono-cultures. Persistent expression of CD31 (a typical large vessel EC marker; +50-fold lower levels than in d12 2D iETV2-ECs) demonstrated that iETV2-ECs persisted until day 32 of co-culture (FIG. 4C). Interestingly, a significant induction of FCGR2B, STAB1, LYVE1 and MRC1 was observed in HepMat co-cultures compared with iETV2-EC-HepMat mono-cultures, even if other LSEC marker genes were not increased (FIGS. 4A and 4C). Compared to PSC-HSC HepMat mono-cultures, fibrotic HSC transcripts decreased further in HepMat co-cultures, with 10-100 fold lower levels of COL1A1, COL3A1, COL5A2, ACTA2 and LOXL2. The 2.5-fold reduction in LRAT levels, might be due to the dilution of HSCs in the mix of other cells (FIG. 4A). However, expression of RGS5 and IGFBP5 remained low (FIG. 4C). Finally, expression of the Mφ marker genes remained relatively unchanged in HepMat co-cultures compared to PSC-Mφ HepMat mono-culture, even if MARCO expression levels were significantly higher in co-cultures than mono-cultures. However, other putative KC marker genes remained unchanged (FIGS. 4A and 4C).

To gain a better understanding of the cellular composition of the co-culture system and to assess expression of each cell type separately rather than on a mixture of cells, H&E and cyclic multiplex immunofluorescence stainings were performed. HepMat co-cultures were fixed on day 32 and embedded without collagenase dissociation to keep the internal structures of the different cell groups visible by light microscopy intact. Histological and MILAN analysis of d32 HepMat co-cultures revealed presence of all different cell types that were embedded (FIGS. 4 D and E). The co-cultures showed characteristics of one coherent functional unit with a balanced distribution of each of the cell types. For example, the epithelial compartment could be subdivided in different stages (of development): hepatocytes (AFP−/ALB+/CYP3A4+ mature as well as AFP+/ALB+/CYP3A4− intermediate hepatocytes, and VIM+/ALB+ mesenchymal hepatocytes), AFP+/KRT19+ hepatic progenitors, and AFP−/KRT19+ cholangiocytes. The ductular structures were clearly positive for cholangiocytic markers, while the larger hepatocyte-like cells clearly expressed hepatocellular markers and were able to store glycogen. Furthermore, immunofluorescence stains revealed the presence of flat CD31+ endothelial cells that were lining the epithelial components. In addition, CD68+ macrophages and VIM+ mesenchymal cells were scattered between these structural components. This was confirmed by further sectioning of additional hydrogels and H&E staining as well as immunostaining, analysed confocal microscopy as shown in.

Thus, the HepMat co-culture system supported maintenance of all four cell populations for at least 32 days, induced maturation of HLCs, induced less fibrogenic PSC-HSCs and an apparent more LSEC committed EC progeny.

### Example 5. HepMat HC3x-HLC, iETV2-EC, PSC-HSC and PSC-Mφ Co-Cultures can be Used to Model Liver Steatosis and Fibrosis

It was investigated whether HepMat co-cultures could be used to model liver fibrosis/steatosis, by exposing cultures either to TGFβ or oleic acid (OA). d32 HepMat co-cultures were treated with 25 ng/ml of TGFβ for an additional 3 days (single administration) or 7 days (2 administrations). In HepMat co-cultures, exposure to TGFβ caused a significant increase in transcripts for COL1A1 (17±3.5-fold) and COL3A1 (13±3.4-fold), but not COL5A2 or LOXL2 (FIG. 5A). TGFβ exposure for 7 days also induced a 7±0.49 and 13±4.3-fold induction of IL1α and IL6 transcripts, respectively (FIG. 5A). Comparatively, when mono-cultures of HC3x-HLCs, PSC-HSCs or PSC-Mφs were exposed to a similar schedule of TGFβ, a significant but much smaller induction of COL1A1 transcripts was observed in HC3x-HLC (9±1.6-fold 3 days treatment; 5±0.7-fold 7 days treatment) and COL1A1 and COL3A1 transcripts in PSC-HSC (4±1.1-fold d3). TGFβ did not induce inflammatory gene expression in Stellate cells and macrophages to the modest level compered to co-cultures. iETV2-ECs did not survive the TGFβ treatment; therefore, no data is shown. In addition, TGFβ induced a 7±0.4-fold higher level of pro-collagen and 17±3.4-fold higher level of IL6 on day 7 of treatment in HepMat co-culture supernatants. Pro-collagen levels were unchanged in HC3x-HLCs and PSC-Mφ mono-cultures and ±3-fold increase in PSC-HSCs following 3 and 7 days of treatment with TGFβ. Hence, fibrogenic responses are more robust when a direct interplay between NPCs and hepatocytes is possible in HepMat co-cultures compared with HC3x-HLC, PSC-HSC- or PSC-Mφ HepMat mono-cultures.

d32 HepMat co-cultures were also exposed to 0.8 mM OA for 3 days (1 dose) and 7 days (2 doses). BODIPY® staining revealed significant lipid accumulation in co-cultures treated with OA already on day 3, which persisted until day 7 (FIG. 6D). In HepMat co-cultures, OA induced a 14±2.3- to 18±1.3-fold induction of COL1A1 and COL1A3 already after 3 days, and an even greater induction of COL1A1, COL1A3, COL5A2 as well as LOXL2 on day 7 (FIG. 6A). Moreover, OA also massively induced IL6 expression (1200-fold d3; 1000-fold d7) and induced a 5±2.3- to 19±3.4-fold induction of TNFα and IL1α on day 3 (FIG. 6A). These responses were much more modest in mono-cultures, with a ±9-fold induction in COL1A1 and COL3A1 transcripts 3 days after OA addition, and a significantly lower induction of inflammatory cytokines in HC3X-HLC and PSC-Mφs mono-cultures. As iETV2-ECs did not survive OA treatment, no data is shown. Consistent with the RT-qPCR data, a 5±0.5 and 12±1.5-fold increase in pro-collagen was observed in supernatants 3 and 7 day HepMat co-cultures after administration of OA, and 90±5-fold increase in IL6 on day 7. By contrast, OA induced no significant changes in either pro-collagen or inflammatory cytokines in supernatants of any of the mono-cultures (FIG. 6B). Thus, the significantly greater induction of both fibrogenic genes and inflammatory cytokine genes (especially IL6) in OA treated HepMat co-cultures compared with the different mono-cultures is consistent with the notion that liver inflammation and fibrosis in response to lipids (as in NASH) requires a direct interplay between NPCs and hepatocytes.

The effect of 2 late phase III anti-NASH drugs was tested, namely the farnesoid X receptor agonist obeticholic acid (OCA), which has shown improvement in fibrosis in 23% of patients vs. placebo treated patients, and the PARα and PPARδ activator, elafibranor (ELN), which was shown to not significantly improve liver fibrosis compared to placebo-treated patients. When HepMat co-cultures were co-treated with either OA & OCA or OA & ELN for 3 days, a significant reduction in bodipy staining was observed. OCA robustly decreased COL1A1 and COL3A1 levels, while ELN only inhibited COL1A1 transcripts; which was translated in a significant albeit not complete inhibition of pro-collagen secretion by OCA and no effect from ELN on pro-collagen concentrations in culture supernatants. A similar effect was observed on proinflammatory parameters. Both OCA and ELN significantly reduced IL6 transcripts, but only OCA could inhibit production of secreted IL6.

### Example 6 Material and Methods

PEG, peptides and synthesis of hydrogel: Vinyl sulfone-functionalised four-arm PEG (four-arm PEG-VS 10K) was purchased from Jenkem, USA. The monocysteine ECM peptides mimics (Ac-GCGYG-peptide-SPG-NH2) [SEQ ID NO: 53] and MMP-sensitive cross-linker peptides (Ac-GCRDE-peptide-EDRCG-NH2) [SEQ ID NO:54] and [SEQ ID NO:55] were purchased from Genscript, USA. Herein the underlined Glycine and Glutamic acids are optional. PEG hydrogels were prepared as described in Lutolf & Hubbel cited above with slight modifications. Four-arm PEG vinyl sulfone was conjugated with monocysteine ECM peptide (250 μM) mimics via a Michael-type addition reaction in 0.3 M HEPES buffer (pH 7,6) at 37° C. for 30 min to make a peptide-PEG precursor solution. The peptide-PEG precursor solution was mixed with cells at a concentration of 3×10⁷ cells/ml. Hydrogel formation was triggered by the addition of MMP-sensitive cross-linker peptides mixed in stoichiometrically balanced ratios to generate hydrogel networks of a desired PEG content at 37° C. for 30 min. Dissociation and release of cellular aggregates grown in PEG for downstream cell processing or re-embedding was accomplished by enzymatic digestion of the gels.

Screening studies: Three properties of the hydrogels were tuned to create unique microenvironments for optimal hepatocyte like cell (HLC) maturation: (1) any combination of six different ECM and cell adhesion molecule (CAM) peptides (Genscript, USA)(Table 1), (2) different MMP-sensitive peptide sequences to tune cell mediated degradability of the hydrogel cell mediated degradability of the hydrogel by (Genscript, USA), and (3) and different PEG polymer concentration to tune hydrogel stiffness. Using a discrete numeric design using the JMP Pro software (SAS Institute), 24 unique combinations of six peptides were created as shown in Table 2, which in combination with the different MMP-sensitive peptide sequences and different mechanical properties resulted in 216 microenvironments that were tested. The initial screen was done with genetically unmodified PSC-hepatoblasts. Specifically, PSC cells were differentiated until day 8 as described, at which time they were encapsulated into hydrogels and allowed to differentiate until day 20/day 32. Determination of cell differentiation was done by RT-qPCR following TRIzol reagent (Thermo Fisher Scientific) treatment to extract the RNA from the embedded cells and -benzyloxy-4-trifluoromethylcoumarin (BFC) metabolisation studies.

Mechanical Characterisation of PEG Hydrogels

The stiffness of PEG hydrogels was determined using a Chiaro Nanoindenter (Optics11, Amsterdam, Netherlands), by applying serial indentations with spherical glass probe (r, 24.5 μm) attached to flexible cantilever (k, 0.063 N/m). Loading and unloading velocities of the probe were set to 1.5 and 15 μm/s, respectively, by applying 2 seconds of holding period in between. For each condition, matrix scans (6×6 points) from two random locations were obtained from replicate hydrogels. Load vs. displacement curves were extracted individually for each indentation point and reduced Elastic Modulus (E) were calculated by using Hertzian Contact Model (Poisson's ratio, 0.5) with Piuma Dataviewer Software (Optics11, Netherlands).

Testing the Efficiency of Peptide Conjugation

The ECM/CAM peptides were replaced by monocysteine 5-FAM peptides. 250, 1000, and 2000 μM of the 5-FAM peptides were reacted with the vinyl sulfonevinylsulfone PEG arms using a Michael-type addition reaction in the same as done for ECM/CAM mimicking peptides. The efficiency of the conjugation reaction was assessed by taking z-stacked images of peptide-conjugated peptides by LSM 880 Confocal microscopy (Zeiss, Germany). Z-stack of 10 planes from three random locations was scanned for each condition, and average fluorescent intensity was calculated by ImageJ Software.

hESC Differentiation to Hepatocyte-Like Cells

The hESC line H9 (WA09) was purchased from WiCell Research Institute (Madison, 15 WI) and expanded feeder free on matrigel (BD biosciences) coated plates in Essential 8 or Essential 8 Flex (Thermo Fisher Scientific). H9 cells were differentiated towards HLCs as described. Briefly, H9 cells were made single cell using accutase and plated on matrigel-coated plates at ±8.75×10⁴ cells/cm² in mTeSR medium. When cells reached 70-80% confluence differentiation was started using the previously described cytokine regimens in liver differentiation medium (LDM) and was stopped after 20 or 40 days of differentiation. All cytokines were purchased from Peprotech (NJ). Differentiation medium was supplemented with 0.6% dimethylsulfoxide (DMSO) during the first 12 days of the culture and with 2.0% DMSO during the last 8 days of differentiation. Genetically modified PSCs (termed HC3x as in Boon et al. was performed in liver differentiation medium (LDM) until D12; after which 3X concentrate of non-essential amino-acids (AAs) was added to the culture until day 14, and from day 14 until the end of the culture, glycine, at a concentration of 20 g/L was added combined with the AAs [Boon et al. cited above]. When AAs were added, DMSO was omitted from the culture.

hESC Differentiation to ETV2-Inducible Endothelial Cells (iETV2-ECs)

The iETV2 cell line was generated by recombining a Tet-inducible cDNA for ETV2 in the FRT-flanked cassette in the AAVFFS1 locus of PSCs. PSCs containing the inducible overexpression cassette for ETV2 were differentiated towards endothelial cells (ECs) using LDM containing 5 μl/ml doxycycline and 10 ng/ml bFGF starting on day 0 of differentiation. From day 2 onwards, 2.0% fetal bovine serum (FBS) was added to the medium. iETV2-ECs were dissociated with (StemPro Accutase Cell dissociation Reagent, Gibco) and passaged every 4 days until day 12, when they were encapsulated in hydrogels.

hESC Differentiation to Hepatic Stellate Cell-Like Cells

Differentiation of PSCs towards hepatic stellate cells (HSCs) were performed as in Coll et al. cited above. H9 cells were grown on Matrigel coated plates until confluency, and then collected as single cells by accutase treatment, and plated on matrigel-coated plates at 5×10⁴ cells/cm² density in mTeSR medium with RevitaCell Supplement (Thermo Fisher Scientific). Differentiation was started when cells reached 70-80% confluency. At the start of differentiation, mTeSR medium was replaced by LDM the different cytokine regimes as described in Coll et al cited above. On day 8, cells were harvested with 0.05% Trypsin (Gibco) treatment and re-plated. On day 12 cells were collected by accutase treatment and encapsulated in the hydrogels.

hESC Differentiation to Macrophages

hESC were differentiated towards macrophages as described in van Wilgenburg et al. cited above. Cells were resuspended at a final cell concentration of 1×10⁵ cells/mL in mTeSR™-1 spin-EB medium (mTeSR™-1, Stem Cell Technologies), 1 mM Rock-inhibitor (Y27632; Calbiochem); BMP-4 (50 ng/ml, Peprotech), SCF (20 ng/mL, Peprotech) and VEGF (50 ng/mL, Peprotech)). 100 μL was added per well was added in 96-well ultra-low adherence plates (Greiner Bio-one) and the plates centrifuged at 300 rcf for 5 min, and incubated for 4 days at 37° C. and 5% CO2. EBs were fed every day by gently aspirating 50 μL medium and gently adding 50 μL of fresh EB medium. On day 4, approx. 20 EBs were transferred into one well of a six-well tissue culture plate in 4 mL medium (X-VIVO™15 (Lonza)) supplemented with glutamax (2 mM, Invitrogen), SCF (50 ng/ml, Peprotech), M-CSF (50 ng/ml, Peprotech), IL-3 (50 ng/ml), FLT3 (50 ng/ml, Peprotech) and TPO (5 ng/ml, Peprotech) 100 U/mL penicillin, 100 μg/mL streptomycin (Invitrogen) and β-mercaptoethanol (0.055 mM, Invitrogen) until day 11 with a media change on day 8. From day 11 onwards, X-VIVO™15 (Lonza) supplemented with glutamax (2 mM,) β-mercaptoethanol (0.055 mM, Invitrogen), FLT3 (50 ng/ml), M-CSF (50 ng/ml) and GM-CSF (25 ng/ml) till the end of differentiation with a media change every week. From day 16 onwards cells were collected for encapsulation in hydrogels either for mono-culture or co-culture studies.

Co-Cultures

The different PSC-liver cell progeny were encapsulated in PEG hydrogels (hepatoblasts harvested on D8; iETV2-ECs on D12; HSCs on day 12 and MEs from day 16 onwards at a ratio of 2:1:1:0.5, respectively. The media used for these co-culture experiments was a combination of LDM and StemPro™-34 SFM medium (1:1) combined with all growth factors/cytokines/additives used for each cell type. The co-culture medium consisted therefore of: LDM+Retinol+Palmitic acid+Stem Pro 34 with the following combinations of cytokines as described in table 4). Encapsulated cells were maintained and differentiated in hydrogels for an additional 32 days.

RNA Extraction and Reverse-Transcription Quantitative PCR (RT-qPCR).

RNA extraction was performed using TRIzol reagent (Invitrogen) following manufacturer's instructions. At least 1 μg of RNA was transcribed to cDNA using the Superscript III First-Strand synthesis (Invitrogen). Gene expression analysis was performed using the Platinum SYBR green qPCR supermix-UDG kit (Invitrogen) in a ViiA 7 Real-Time PCR instrument (Thermo Fisher Scientific, Waltham, MA). The ribosomal protein L19 transcript (RPL19) was used as a housekeeping gene for normalization.

Histology

Hydrogels were fixed with 4% (w/v) paraformaldehyde (PFA, Sigma-Aldrich) overnight at 4° C., washed 3 times with PBS and submerged in PBS-sodium azide (0.01% v/v) solution at 4° C. until embedded in paraffin. Hydrogel sections (5 μm) were prepared using a microtome (Microm HM 360, Marshall Scientific.) For Hematoxylin and Eosin (H&E) and Periodic-acid Schiff (PAS) staining, sections were treated with xylene solution to remove the paraffin, and gradually rehydrated in ethanol (100 to 70%, v/v). H&E staining was performed by submerging rehydrated hydrogel sections in Harris Hematoxylin solution, acid alcohol, bluing reagent and Eosin-Y solution by order. Stained samples were dehydrated with ascending alcohol series, washed in xylene solution, and mounted with DPX mountant (Sigma). PAS and staining were performed according to the manufacturer's instructions.

Immunofluorescence Analysis.

Following deparaffinisation in xylene and rehydration in descending alcohol series, heat-mediated antigen retrieval was performed by incubating hydrogel sections in Dako antigen retrieval solution (Dako, Copenhagen, Denmark) for 20 min at 98° C. This step was followed by cell permeabilisation with 0.01% (v/v) Triton-X (Sigma-Aldrich) solution in PBS, for 20 minutes. Samples were then incubated with 5% (v/v) Goat or Donkey Serum (Dako, Copenhagen, Denmark) for 30 min. Primary antibodies diluted in Dako antibody diluent solution, were incubated overnight at 4° C., followed by washing steps and incubation with Alexa-coupled secondary antibody (1:500) and Hoechst 33412 (1:500) solution for 1 hour at room temperature. Finally, samples were washed in PBS, and mounted with Vectashield antifade mounting medium (Vector Laboratories). Stained sections were imaged using laser scanning confocal microscope (LSM 880, Zeiss, Germany), and image processing were performed on ZEN Blue software (Zeiss, Germany).

Flow Cytometry.

Cells present in HepMat cultures were isolated by collagenase treatment of 6 hydrogels. Isolated clusters were washed with 1×PBS and dissociated to single cells by treatment with TrypLE™ Express Enzyme (1×), phenol red (GIBCO, Thermo Fisher Scientific) for 20 min. After a PBS wash, single cells were stained with the primary antibodies for 45 min along with respective isotype controls. Dead cells were excluded by propidium iodide staining. All samples were analysed using a FACS-Canto (BD). As isotype control, rabbit IgG (BD Pharmingen) was used.

Functional Assessments

Hepatic proceny: To assess glycogen storage, sections were stained using periodic acid-Schiff (PAS, Sigma). CYP3A4 dependent metabolisation over 4 h was determined using the fluorimetric probe BFC. Albumin secretion rate was quantified using the human albumin ELISA quantitation kit (Bethyl Laboratory, USA).

TGFβ exposure of HepMat-PSC-HSC cultures: Day 32 HepMat-PSC-HSC mono-cultures hydrogels were exposed to 25 ng/ml of TGFβ (Peprotech) on day 32. Expression of inflammatory and fibrogenic genes was determined by RT-1PCR. Supernatants were collected for pro-collagen measurement by ELISA.

LPS exposure of HepMat-PSC-MEs cultures: Day 32 HepMat-PSC-M□s mono-cultures were exposed to 100 ng/ml of LPS (Sigma-Aldrich). Expression of inflammatory genes was determined by RT-qPCR. Supernatants were collected for inflammatory cytokine measurement by ELISA.

TGFβ exposure of HepMat-co-cultures and mono-cultures: Day 32 HepMat co-cultures and dayy 32 HepMat HC3x HLC, HSC and Mφ mono-cultures were incubated with 25 ng/ml TGFβ for 7 days (addition on day 0 and day 3). Expression of inflammatory and fibrogenic genes was determined by RT-qPCR. Supernatants were collected for pro-collagen measurement by ELISA.

Oleic acid (OA) exposure of HepMat-co-cultures and mono-cultures: Day 32 HepMat co-cultures and Day 32 HepMat HC3x HLC, HSC and Mφ mono-cultures were incubated with 800 μM of OA (Sigma-Aldrich) for 7 days (addition on day 0 and day 3). Day 32 HepMat co-cultures were also incubated with a combination 800 μM of OA and 1 μM Obeticholic acid (OCA) (Company) or 800 μM of OA and 30 μM Elafibranor (ELN) (Company) for 3 days (addition on day 0 only). Expression of inflammatory and fibrogenic genes was determined by RT-1PCR. Supernatants were collected for pro-collagen measurement by ELISA on day 3 and day 7. In addition, co-culture were assayed for presence of steatosis by BODIPY® staining on day 3 and day 7 as follows: Gels were transferred to Glass Bottom plates (cellvis) for live-cell imaging, following incubation with BODIPY® 493/503 for lipids (Thermo Fisher Scientific) and Hoechst (Sigma Aldrich). Sample was visualised and scanned on a laser scanning confocal microscope (LSM 880, Zeiss, Germany),

TNF-Alpha, IL1alpha, IL6 and Pro-Collagen 1 ELISA

The supernatant was assayed for human TNF-alpha, ILla1pha, and IL6 by ELISA (Biolegend, CA, USA), according to the manufacturer's instructions. Presence of pro-collagen type I was detected by Pro-collagen Type I C-peptide ELISA kit (Takara Bio Inc). Secreted level of TNF-alpha, IL1alpha, IL6 and pro-collagen 1 were normalised for the cell number.

Quantification and Statistical Analysis

Results are expressed as the arithmetic mean±standard error of the mean (SEM). All experimental results are from a minimum of 3 biological replicate experiments unless otherwise stated. Statistical comparisons between groups were done using Student's t test, one way Anova or two way Anova test when appropriate. A p-value of <0.05 was considered significant. Analyses were carried out using either JMP pro software (SAS, Institute, USA) or GraphPad Prism 8.0 (GraphPad prism Software Inc., La Jolla, CA).

1. A composition comprising a three-dimensional network of a synthetic hydrogel covalently cross-linked with at least 6 different peptides, each peptide comprising a metalloproteinase (MMP) cleavable linker and fragment from an adhesion domain of an Extra Cellular Matrix (ECM) component or from a Cell-Adhesion molecule (CAM),
wherein said at least six different peptides comprise:
a peptide from fibronectin,
a peptide from collagen I,
a peptide from collagen IV, other than the collagen IV fragment FYFDLR [SEQ ID NO:17],
a peptide from Collagen III, other than collagen III fragment GLKGEN [SEQ ID NO:15],
a peptide from laminin,
and wherein said at least six peptides do not comprise a peptide from perlecan, from elastin or from tenascin,
and do not comprise the peptide HAVDI [SEQ ID NO:22] of N cadherin.

2. The composition according to claim 1, wherein said six different peptides comprise respectively the sequence of:
[SEQ ID NO:5], [SEQ ID NO:8], [SEQ ID NO:12], [SEQ ID NO:13], [SEQ ID NO:16] and [SEQ ID NO:23] (pool PP15), or
RGD, [SEQ ID NO:7], [SEQ ID NO:11], [SEQ ID NO:14], [SEQ ID NO:16] and [SEQ ID NO:23] (pool PP16), or
[SEQ ID NO:5], [SEQ ID NO:9], [SEQ ID NO:12], [SEQ ID NO:14], [SEQ ID NO:16] and [SEQ ID NO:21] (pool pp17), or
[SEQ ID NO:2], [SEQ ID NO:9], [SEQ ID NO:12], [SEQ ID NO:13], [SEQ ID NO:16] and [SEQ ID NO:24] (pool pp18), or
[SEQ ID NO:6], [SEQ ID NO:10], [SEQ ID NO:11], [SEQ ID NO:13], [SEQ ID NO:16] and [SEQ ID NO:21] (pool pp8).

3. The composition according to claim 1 or 2, wherein said six different peptides comprise respectively the sequence of [SEQ ID NO:5], [SEQ ID NO:8], [SEQ ID NO:12], [SEQ ID NO:13], [SEQ ID NO:16] and [SEQ ID NO:23] (pool PP15).

4. The composition according to any one of claims 1 to 3, wherein the synthetic hydrogel is a 4-armed polyethylene glycol PEG with Mr of 10.000 kD.

5. The composition according to any one of claims 1 to 4, wherein the metalloproteinase (MMP) cleavable linker comprises the sequence GPQGIAGQ [SEQ ID NO:52].

6. A composition in accordance with any one of claims 1 to 5, and further comprising PSC differentiated into hepatocyte-like cells and optionally one or more of hepatic stellate cells, liver sinusoidal endothelial cells and liver macrophages.

7. The composition according to claim 6, wherein
the hepatocyte like cells metabolise 7-Benzyloxy-4-triFluoromethyl-Coumarin (BFC), or express one or more of HNF4a, HNF6, PROX1, CYP3A4, CYP2D6, PEPCK, G6PC and PGC1a.
the hepatic stellate cells express one or more of PDGFR-alpha, DESMIN, LRAT, RGS5, COL3A1, COL1A1, LOXL2 and NCAM,
the liver sinusoidal endothelial cells express one or more of CD31, FCGR2B, STAB1, FCN3, OIT3 and CLEC4G, and
the liver macrophages express one or more of MARCO, CD5L, SIGLEC1, CD163, CD45, CD68, CD14.

8. The composition according to claim 6 or 7, wherein said cells are aggregated into spheroids.

9. Use of a composition in accordance to any one of claims 6 to 8, for toxicity testing of compounds or conditions applied on said cells.

10. Use of a composition in accordance to any one of claims 1 to 5, for the cultivation of PSC differentiated into hepatocyte-like cells and cultivation of optionally one or more of hepatic stellate cells, liver sinusoidal endothelial cells and liver macrophages.

11. A method of identifying a matrix suitable for cell growth of PSC cells differentiated into hepatocyte like cells, comprising the steps of:
Providing a cell culture medium comprising an artificial matrix, wherein said matrix comprises at least 4 different peptides of an adhesion domain from an Extra Cellular Matrix (ECM) component or from an Cell-Adhesion molecule (CAM), covalently linked to said hydrogel, wherein the peptides further comprise a MMP cleavable sequence,
Growing PSC cells in said medium under conditions allowing the differentiation of said PSC cells into hepatocyte cells,
testing a one or more parameters indicative of a hepatocyte like phenotype,
based on said one or more parameters, identifying a matrix as suitable for cultivation of hepatocyte like cells differentiated from PSC.

12. The method according to claim 11, wherein
the ECM component is selected from the group consisting of Fibronectin, Collagen I, Collagen III, Collagen IV, Laminin, Perlecan, tenascin and Elastin, and
wherein the CAM is E-cadherin or N-cadherin.

13. The method according to claim 11 or 12 and further comprising the steps of:
growing one or more of hepatic stellate cells, liver sinusoidal endothelial cells and liver macrophages and testing one or more parameters indicative for the phenotype of said cell, and,
based on said one or more parameters, identifying a matrix as suitable for co-cultivation of hepatocyte like cells with one or more of hepatic stellate cells, liver sinusoidal endothelial cells and liver macrophages.

14. The method according to claim 13 wherein:
a parameter indicative of a hepatocyte like phenotype is the metabolisation of 7-Benzyloxy-4-triFluoromethyl-Coumarin (BFC), or the expression of one or more of HNF4a, HNF6, PROX1, CYP3A4, CYP2D6, PEPCK, G6PC and PGC1a,
a parameter indicative of hepatic stellate cells is the expression of one or more of PDGFR-alpha, DESMIN, LRAT, RGS5, COL3A1, COL1A1, LOXL2 and NCAM,
a parameter indicative of liver sinusoidal endothelial cells is expression of one or more of CD31, FCGR2B, STAB1, FCN3, OIT3 and CLEC4G, and,
a parameter indicative of liver macrophages is expression of one or more of MARCO, CD5L, SIGLEC1, CD163, CD45, CD68, CD14.


SEQUENCE LISTING (55 sequences)

SEQ ID NO: 1 - protein, 5 aa, Homo Sapiens
GCGYG

SEQ ID NO: 2 - protein, 4 aa, Homo Sapiens
RGDS

SEQ ID NO: 3 - protein, 6 aa, Homo Sapiens
KQAGDV

SEQ ID NO: 4 - protein, 4 aa, Homo Sapiens
REDV

SEQ ID NO: 5 - protein, 5 aa, Homo Sapiens
PHSRN

SEQ ID NO: 6 - protein, 6 aa, Homo Sapiens
KRLDGS

SEQ ID NO: 7 - protein, 6 aa, Homo Sapiens
KGHRGF

SEQ ID NO: 8 - protein, 5 aa, Homo Sapiens
YIGSR

SEQ ID NO: 9 - protein, 5 aa, Homo Sapiens
IKVAV

SEQ ID NO: 10 - protein, 5 aa, Homo Sapiens
IKLLI

SEQ ID NO: 11 - protein, 4 aa, Homo Sapiens
DGEA

SEQ ID NO: 12 - protein, 15 aa, Homo Sapiens
GTPGPQGIAGQRDVV

SEQ ID NO: 13 - protein, 6 aa, Homo Sapiens
  misc_feature: X is hydroxyproline
  misc_feature (3)..(3): Xaa can be any naturally occurring amino acid
GFXGER

SEQ ID NO: 14 - protein, 6 aa, Homo Sapiens
  misc_feature: X is hydroxyproline
  misc_feature (3)..(3): Xaa can be any naturally occurring amino acid
GLXGEN

SEQ ID NO: 15 - protein, 6 aa, Homo Sapiens
GLKGEN

SEQ ID NO: 16 - protein, 12 aa, Homo Sapiens
TAGSCLRKFSTM

SEQ ID NO: 17 - protein, 6 aa, Homo Sapiens
FYFDLR

SEQ ID NO: 18 - protein, 17 aa, Homo Sapiens
TWSKVGGHLRPGIVQSG

SEQ ID NO: 19 - protein, 4 aa, Homo Sapiens
VAPG

SEQ ID NO: 20 - protein, 9 aa, Homo Sapiens
VAEIDGIEL

SEQ ID NO: 21 - protein, 10 aa, Homo Sapiens
LFSHAVSSNG

SEQ ID NO: 22 - protein, 5 aa, Homo Sapiens
HAVDI

SEQ ID NO: 23 - protein, 6 aa, Homo Sapiens
ADTPPV

SEQ ID NO: 24 - protein, 20 aa, Homo Sapiens
LRRFSTMPFMFCNINNVCNF

SEQ ID NO: 25 - protein, 9 aa, Artificial Sequence
  fusion peptide
GCGYGRGDG

SEQ ID NO: 26 - protein, 10 aa, Artificial Sequence
  fusion peptide
GCGYGRGDSG

SEQ ID NO: 27 - protein, 12 aa, Artificial Sequence
  fusion peptide
GCGYGKQAGDVG

SEQ ID NO: 28 - protein, 10 aa, Artificial Sequence
  fusion peptide
GCGYGREDVG

SEQ ID NO: 29 - protein, 11 aa, Artificial Sequence
  fusion peptide
GCGYGPHSRNG

SEQ ID NO: 30 - protein, 12 aa, Artificial Sequence
  fusion peptide
GCGYGKRLDGSG

SEQ ID NO: 31 - protein, 12 aa, Artificial Sequence
  fusion peptide
GCGYGKGHRGFG

SEQ ID NO: 32 - protein, 14 aa, Artificial Sequence
  fusion peptide
GCGYGDPGYIGSRG

SEQ ID NO: 33 - protein, 11 aa, Artificial Sequence
  fusion peptide
GCGYGIKVAVG

SEQ ID NO: 34 - protein, 11 aa, Artificial Sequence
  fusion peptide
GCGYGIKLLIG

SEQ ID NO: 35 - protein, 10 aa, Artificial Sequence
  fusion peptide
GCGYGDGEAG

SEQ ID NO: 36 - protein, 20 aa, Artificial Sequence
  fusion peptide
GCGYGTPGPQGIAGQRDVVG

SEQ ID NO: 37 - protein, 11 aa, Artificial Sequence
  X is hydroxyproline
  misc_feature (7)..(7): Xaa can be any naturally occurring amino acid
GCGYGFXGERG

SEQ ID NO: 38 - protein, 11 aa, Artificial Sequence
  fusion peptide
  misc_feature (7)..(7): Xaa can be any naturally occurring amino acid
GCGYGLXGENG

SEQ ID NO: 39 - protein, 11 aa, Artificial Sequence
  fusion peptide
GCGYGLKGENG

SEQ ID NO: 40 - protein, 18 aa, Artificial Sequence
  fusion peptide
GCGYGTAGSCLRKFSTMG

SEQ ID NO: 41 - protein, 12 aa, Artificial Sequence
  fusion peptide
GCGYGFYFDLRG

SEQ ID NO: 42 - protein, 23 aa, Artificial Sequence
  fusion peptide
GCGYGTWSKVGGHLRPGIVQSGG

SEQ ID NO: 43 - protein, 10 aa, Artificial Sequence
  fusion peptide
GCGYGVAPGG

SEQ ID NO: 44 - protein, 15 aa, Artificial Sequence
  fusion peptide
GCGYGVAEIDGIELG

SEQ ID NO: 45 - protein, 16 aa, Artificial Sequence
  fusion peptide
GCGYGLFSHAVSSNGG

SEQ ID NO: 46 - protein, 11 aa, Artificial Sequence
  fusion peptide
GCGYGHAVDIG

SEQ ID NO: 47 - protein, 12 aa, Artificial Sequence
  fusion peptide
GCGYGADTPPVG

SEQ ID NO: 48 - protein, 26 aa, Artificial Sequence
  fusion peptide
GCGYGLRRFSTMPFMFCNINNVCNFG

SEQ ID NO: 49 - protein, 15 aa, Homo Sapiens
GCRDVPLSYSGDRCG

SEQ ID NO: 50 - protein, 16 aa, Homo Sapiens
GCRDGPQGIAGQDRCG

SEQ ID NO: 51 - protein, 16 aa, Homo Sapiens
GCRDGPQGIWGQDRCG

SEQ ID NO: 52 - protein, 8 aa, Homo sapiens
GPQGIAGQ

SEQ ID NO: 53 - protein, 5 aa, Artificial Sequence
  ECM mimic
  SITE (5)..(5): This residue is C terminally linked to a peptide fragment of an ECM protein and an SPG-NH2 tripeptide
  MOD_RES (5)..(5): ACETYLATION
  VARIANT (5)..(5): may be absent
GCGYG

SEQ ID NO: 54 - protein, 5 aa, Artificial Sequence
  ECM fragments
  MOD_RES (1)..(1): ACETYLATION
  VARIANT (5)..(5): may be absent
  SITE (5)..(5): This residue is linked C-terminally to a fragment of an ECM protein and the peptide of SEQ ID NO55
GDRDE

SEQ ID NO: 55 - protein, 5 aa, Artificial Sequence
  ECM fragment
  SITE (1)..(1): This residu is N terminally linked to a peptide having the sequence of SEQ ID NO54 and a fragment of an ECM protein
  VARIANT (1)..(1): may be absent
  MOD_RES (5)..(5): AMIDATION
EDRCG